(19) United States
(12) Patent Application Publication
(10) Pub. No.: US 2007/0173517 A1
(43) Pub. Date: Jul. 26, 2007

(54) Synthesis of 6,7-Dihydro-5H-imidazo[1,2-a]imidazole-3-sulfonic acid amides

(76) Inventors: Thomas WIRTH, Stadecken-Elsheim (DE); Jutta KROEBER, Bingen-Dietersheim (DE); Thomas NICOLA, Ingelheim am Rhein (DE); Armin RAPP, Bingen-Dromersheim (DE); Dieter GUTHEIL, Bad Kreuznach (DE); Simone A. ORLICH, Schoeneberg (DE); Xiao-jun WANG, Ridgefield, CT (US); Dhileepkumar KRISHNAMURTHY, Ridgefield, CT (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/623,447

(22) Filed: Jan. 16, 2007

Related U.S. Application Data
(60) Provisional application No. 60/743,156, filed on Jan. 20, 2006.

(51) Int. Cl. A61K 31/506; A61K 31/454; A61K 31/4188; C07D 487/04

(52) U.S. Cl. 514/256; 514/322; 514/393; 544/333; 546/199; 548/303.1

(57) ABSTRACT

Disclosed is an improved multi-step process for preparing a compound of Formula I: 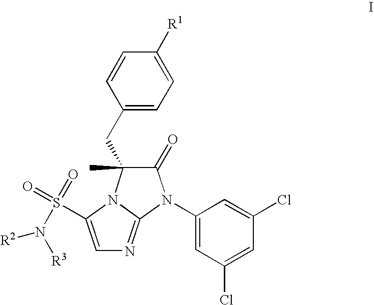
wherein R¹ to R³ are as defined herein. The compounds of formula I inhibit the binding of human intercellular adhesion molecules to the Leukointegrins. As a result, these compounds are useful in the treatment of inflammatory and immune cell-mediated diseases.

This application claims benefit from U.S. Provisional Application No. 60/743,156, filed on Jan. 20, 2006.

### TECHNICAL FIELD

The invention relates to an improved process for the preparation of 6,7-dihydro-5H-imidazo[1,2-a]imidazole-3-sulfonic acid amides useful as agents for the treatment of inflammatory and immune-cell mediated diseases.

### BACKGROUND OF THE INVENTION

6,7-Dihydro-5H-imidazo[1,2-a]imidazole-3-sulfonic acid amides of formula I below, wherein R¹ to R³ are as defined herein, have been reported as small molecule inhibitors of the binding of human intercellular adhesion molecules, including ICAM- 1, ICAM-2 and ICAM-3, to the Leukointegrins, especially CD18/CD11a. As a result, these small molecules are useful in the treatment of inflammatory and immune cell-mediated diseases (See U.S. Pat. Nos. 6,492,408, 6,844,360, WO 2004/041827 A2, U.S. Pat. No. 6,852,748, and WO 2004/041273 A1).

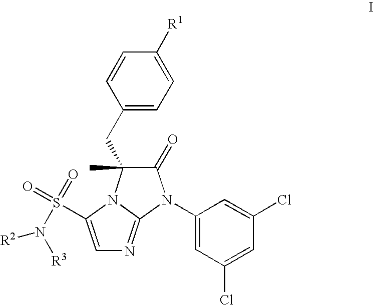

A synthetic route that was used to prepare compounds of formula I (U.S. Pat. No. 6,492,408) is shown in Scheme 1. As illustrated in Scheme 1, reaction of amino-esters of formula II with 3,5-dichlorophenylisothiocyanate provided thiohydantoin III. A solution of triphenylphosphine was treated with azide IV, and the resulting intermediate was reacted with thiohydantoin III to provide guanidine derivative V. Treatment of V with trifluoroacetic acid provided VI. Iodination of VI with N-iodosuccinimide provided VII. Treatment of VII with cyclopentylmagnesium bromide was followed by addition of sulfur dioxide to provide an intermediate magnesium sulfinate salt. Treatment of this intermediate salt with N-chlorosuccinimide provided sulfonyl chloride VIII. Treatment of VIII with the appropriate amine provided the desired compound of formula I or a precursor that could be further modified to provide the desired compound.

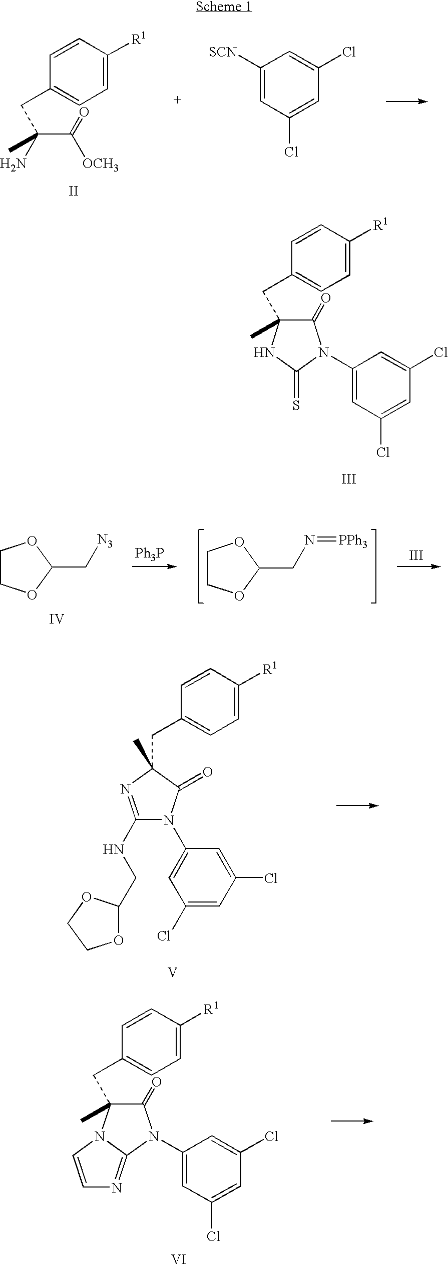

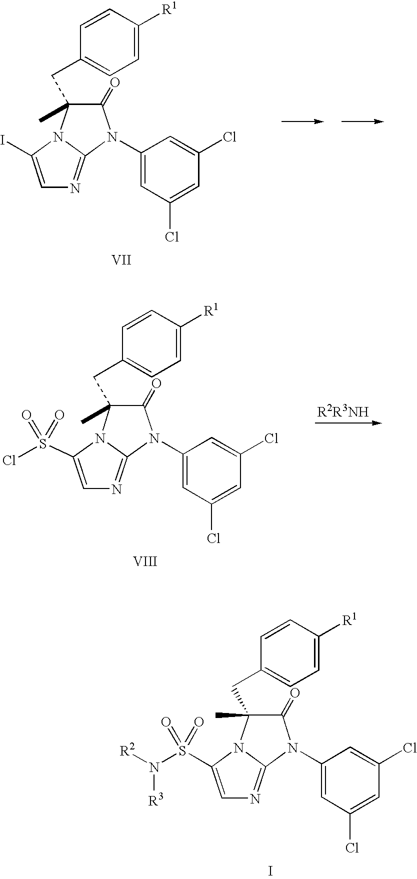

An alternate synthesis of intermediate VII illustrated in Scheme 2 was described in U.S. Pat. No. 6,414,161:

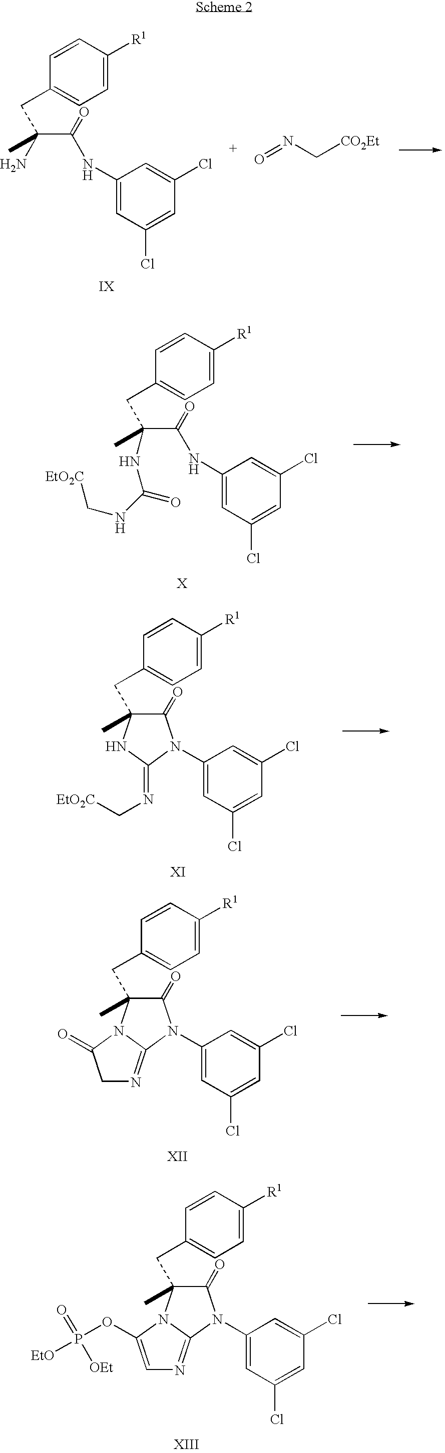

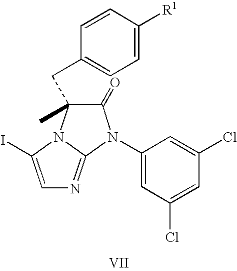

As illustrated in Scheme 2, reaction of amino-amide IX with ethyl isocyanatoacetate provided urea X. Dehydration-cyclization of X with carbon tetrachloride, triphenylphosphine and triethylamine produced guanidine XI. Treatment of XI with trimethylaluminum provided lactam XII. Reaction of lactam XII with ethyl chlorophosphate and bis(trimethylsilyl)amide provided phosphate XIII. Iodination of XIII with trimethylsilyl chloride and sodium iodide provided iodo intermediate VII. Disadvantages of the above two procedures include the use of potentially hazardous reagents such as azide IV (Scheme 1) and the requirement of chromatographic purification, such as purification of XII (Scheme 2). Therefore, the synthetic methods outlined above are not suitable for large scale preparation of compounds of formula I.

Furthermore according to U.S. patent application Ser. No. 11/188,377, now U.S. Application Publication No. 2006/0025447 A1, we have provided an alternate synthesis of above-mentioned compound I as illustrated in the following Scheme 3: 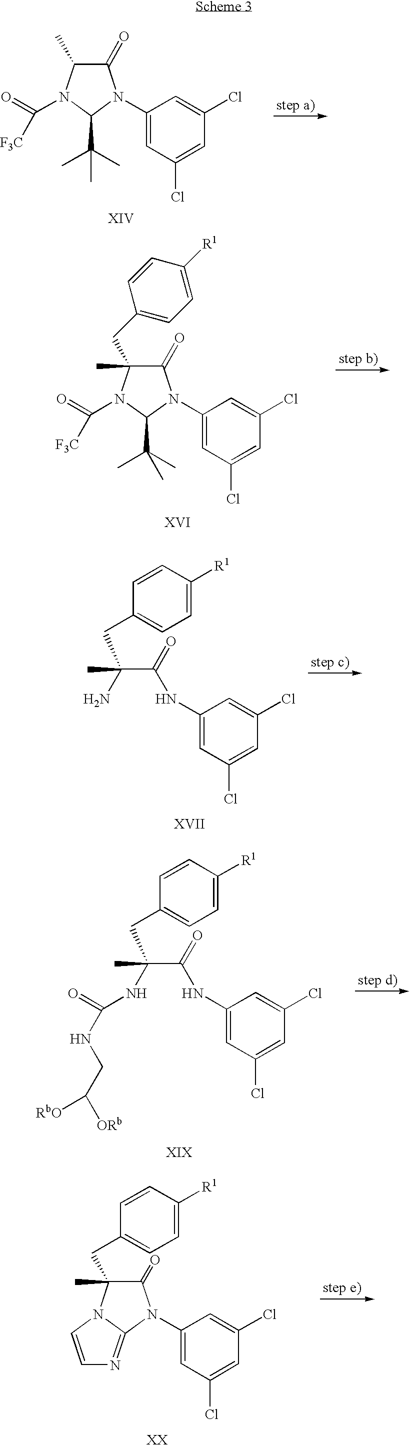 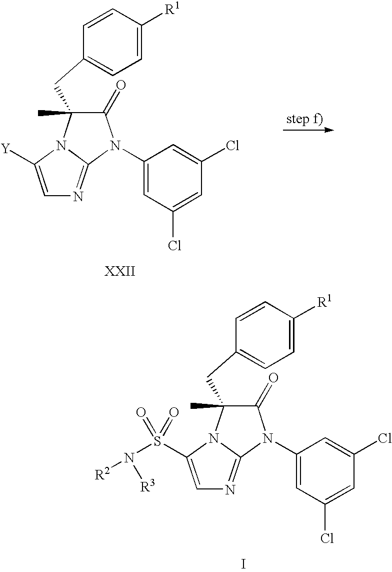
wherein:
R¹ is selected from bromo, trifluoromethoxy, cyano and pyrimidin-5-yl optionally mono- or disubsituted by NH₂;
R^{b} is C₁₋₄ alkyl;
Y is a halogen atom, preferably Br, I; and
R² and R³ are as defined herein.

As illustrated in Scheme 3, the reaction of an imidazolidine compound of formula XIV resulted in an amino-amide compound of formula XVII via a compound of formula XVI and the reaction of the amino-amide compound of formula XVII with a carbamate provided an urea compound of formula XIX. A dehydration-cyclization of the urea compound of formula XIX produced an imidazole compound of formula XX. Halogenation of the compound of formula XX provided the halo intermediate of formula XXII which may be further reacted to the product of the title of formula I.

Therefore the reaction sequence is summarized as follows:
process step a) compound of formula XIV→compound of formula XVI;
process step b) compound of formula XVI→compound of formula XVII;
process step c) compound of formula XVII→compound of formula XIX;
process step d) compound of formula XIX→compound of formula XX;
process step e) compound of formula XX→compound of formula XXII; and
process step f) compound of formula XXII→compound of formula I.

A disadvantage of the above process is that the synthesis method is not totally optimized for large scale preparation of compounds of formula I.

It is therefore an object of the present invention to optimize the process of the preparation of 6,7-Dihydro-5H-imidazo[1,2-a]imidazole-3-sulfonic acid amides taking specially into consideration the aspects of problems of synthesis in large scale, such as safety, quality, operation efficiency, environmental compatibility, economics and costs.

A further object of the present invention is to provide a scalable and simpler process of the preparation of 6,7-Dihydro-5H-imidazo[1,2-a]imidazole-3-sulfonic acid amides as well as a scalable and simpler process of the production of synthesis intermediates thereof, wherein at least one process step of the multi-step process being improved such that a better yield of the produced product is obtained.

A further object of the present invention is to provide a purer end product as well as purer intermediate products in the multi-step or a single step process thereof, which may be isolated easier and faster compared with the prior art processes.

### SUMMARY OF THE INVENTION

The present invention is directed to an improved process for the preparation of compounds of formula I (step a) to step f)); improvements are realized in step c) and/or step e) and/or step f). This improved process is optimized in practical and economical aspects and involves fewer chemical steps while no chromatographic purification is necessary. The advantages of the known processes of the preparation of 6,7-Dihydro-5H-imidazo[1,2-a]imidazole-3-sulfonic acid amides as shown in scheme 3 have been maintained while additionally process step c) and/or step e) and/or step f) have been significantly improved.

One aspect of the invention is directed to a process for preparing compounds of formula I: 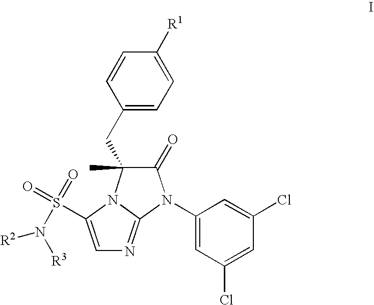
wherein
R¹ is selected from bromo, trifluoromethoxy, cyano and pyrimidin-5-yl optionally mono- or disubsituted by NH₂; and
R² and R³ are each independently selected from the group consisting of
   a) hydrogen; and
   b) a C₁₋₄ straight or branched alkyl group, optionally mono- or disubstituted with moieties independently selected from oxo, —OH, NH₂ and —C(O)NR⁴R⁵, wherein R⁴ and R⁵ are independently selected from:
      (1) hydrogen, and
      (2) a C₁₋₄ straight or branched alkyl group which alkyl group is mono- or disubstituted with moieties independently selected from CONH₂ and OH;
         or
R² and R³, combined with the nitrogen they are bonded to, form:
   (1) a pyrrolidine or piperidine ring, each optionally substituted with the group —C(O)NR⁶R⁷, wherein R⁶ and R⁷ are independently selected from
      a) hydrogen; and
      b) a C₁₋₄ straight or branched alkyl group, optionally mono- or disubstituted with moieties independently selected from oxo, —OH and NH₂;
   (2) a morpholine ring; or
   (3) a piperazine ring;
      or a pharmaceutically acceptable salt thereof.

The process comprises the following steps (wherein, unless otherwise defined, all the substituent groups in the chemical formulas depicted in the synthetic steps hereafter have the same definitions as set forth above for formula I):
a) reacting a compound of formula XIV and a compound of formula XV in the presence of a strong base at a temperature from 0° C. to ambient temperature, in an aprotic organic solvent, to provide a compound of formula XVI: 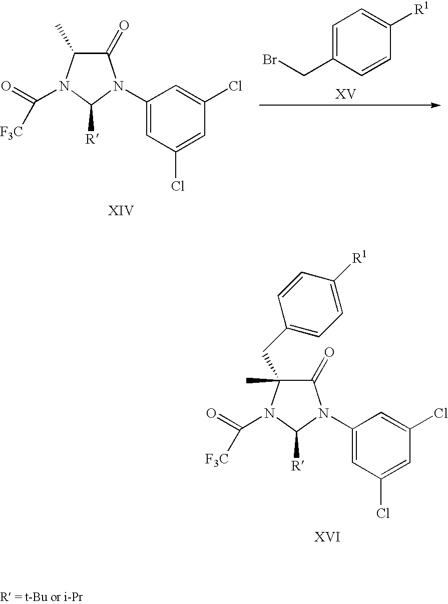
b) deprotecting and hydrolyzing a compound of formula XVI produced in step a) by treating the compound of formula XVI with a base and optionally (and preferably) in addition with a phase transfer catalyst in tetrahydrofuran or 2-methyl tetrahydrofuran, followed by an acid to form a compound of formula XVII: 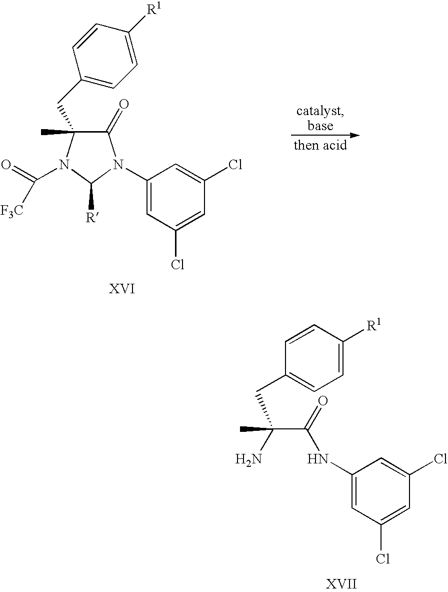
c) reacting the compound of formula XVII produced in step b) with a compound of formula XVIII, wherein R^{a} is aryl and R^{b} is C₁₋₄ alkyl, and an organic base in a polar organic solvent to form a compound of formula XIX. 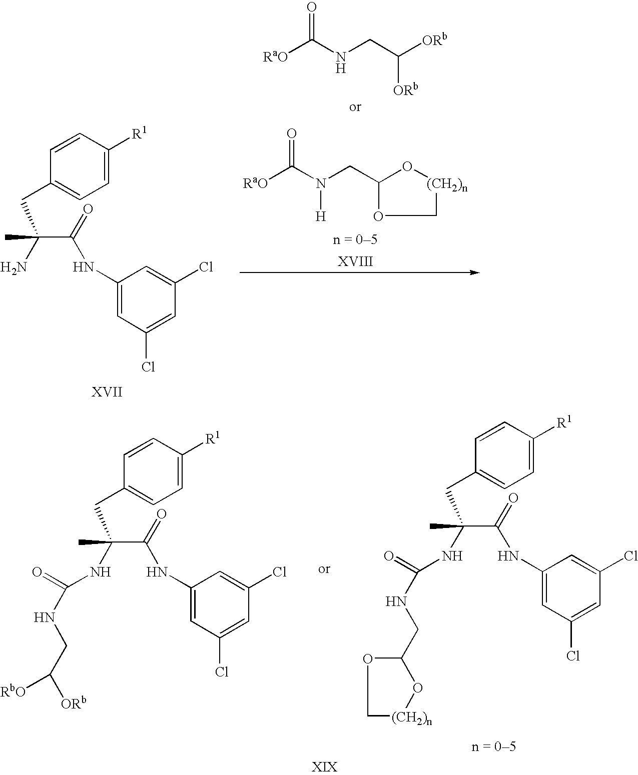
d) reacting the compound of formula XIX produced in step c) with a compound of formula (R^{c})₃P, wherein R^{c} is C₁₋₄ alkyl, C₃₋₆ cycloalkyl or aryl, a carbon tetrahalide and a tri-C₁₋₆ alkylamine in an aprotic organic solvent, followed by adding an acid to form a compound of formula XX, or
d) alternatively, reacting a compound of formula XIX produced in step c) with a compound of formula (R^{c})₃PX₂, wherein R^{c} is C₁₋₄ alkyl, C₃₋₆ cycloalkyl or aryl, and X is a halide, and a tri-C₁₋₆ alkylamine, in an aprotic organic solvent, followed by adding an acid to form a compound of formula XX, or
d) alternatively, reacting a compound of formula XIX produced in step c) with a boronic acid compound ArB(OH)₂, wherein Ar is an aromatic carbocyclic group substituted with one or more electron withdrawing groups, in an aprotic organic solvent to form a compound of formula XX: 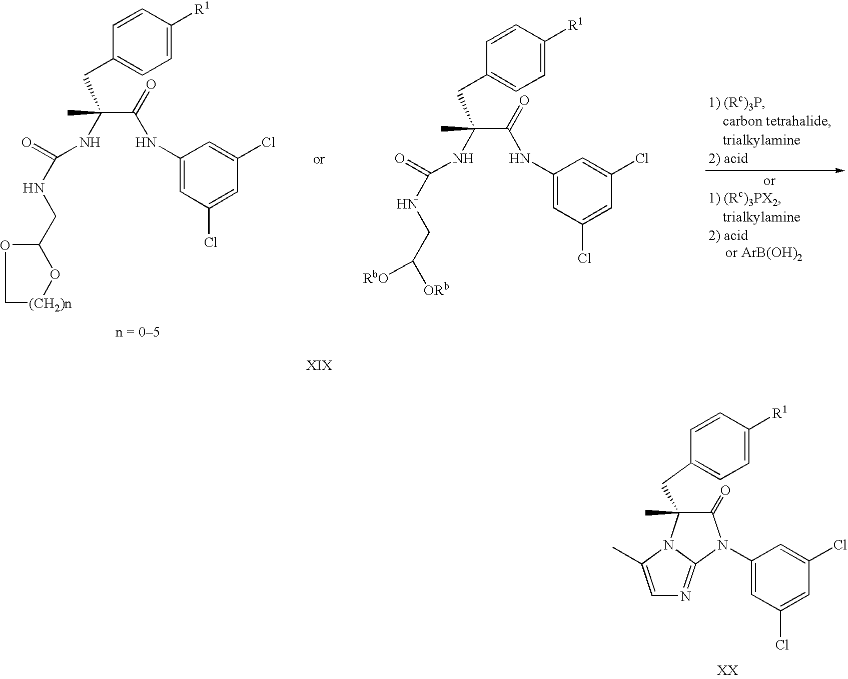
e) reacting the compound of formula XX produced in step d) with a compound of formula XXI (a compound of formula XXI-1 or alternatively a compound of formula XXI-2), wherein Y is a halogen, in an aprotic organic solvent to form a compound of formula XXII: 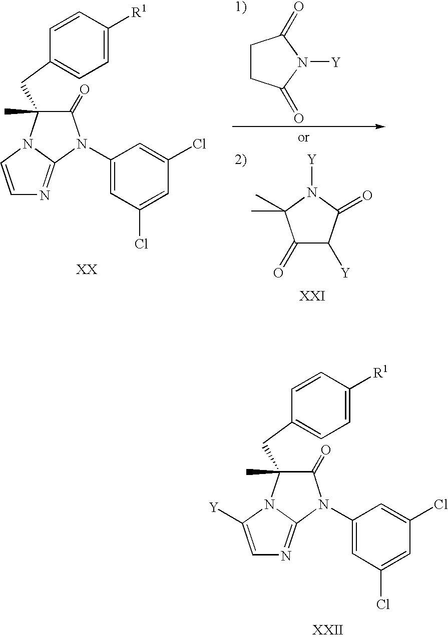
f) reacting the compound of formula XXII produced in step e) with a compound of formula R^{d}MgY, wherein R^{d} is C₁₋₆ alkyl or C₃₋₆ cycloalkyl and Y is halogen, sulfur dioxide and N-chlorosuccinimide, followed by a base and a compound of formula XXIII in an aprotic organic solvent to form a compound of formula I, without isolation of intermediates formed during this step:

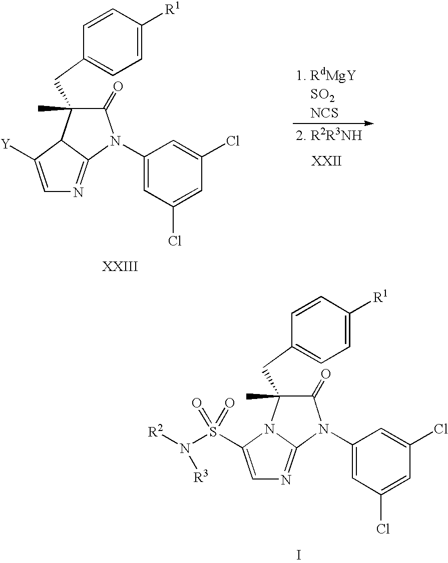

The final compounds of formula I can be converted to its pharmaceutically acceptable salts using any conventional techniques known in the art.

An improvement according to the present invention is provided in process step c), wherein a compound of formula XVII is reacted to provide a compound of formula XIX. The improvement of the above-mentioned process is that the organic solvent as usually required to be present in step c) is omitted, i.e. the base used in the reaction simultaneously serves as solvent so that the base fulfills two functions, namely the function of a basic compound and the function of a solvent.

A further improvement according to the present invention may be preferably provided in process step c), wherein a compound of formula XVII is reacted to provide a compound of formula XIX. The improvement of step c) may be to perform the crystallization of the product in a solvent system selected from an alcohol/water system.

Still a further improvement of the above process may be preferably provided in step e), wherein a compound of formula XX is reacted to provide a compound of formula XXII, the halogenation agent should be preferably selected in such a manner that it is hardly or slightly soluble in the solvent used. Furthermore it is preferred to add the halogenation agent preferably in solid form, more preferably in portions, to the educt in step e) in a solvent comprising a compound having the general formula XX.

Another improvement of the above process may be preferably provided in step f), wherein a compound of formula XXII is reacted to provide a compound of formula I, which may be divided in the sub-step 1, which represents an N-chlorosuccinimide oxidation, sub-step 2, which represents the sulfamidation and optionally sub-step 3, which represents the crystallization of the crude product of formula I. The improvement may be performed in one, two or all three sub-steps.

In sub-step 1 the solvent of N-chlorosuccinimide may be preferably modified such that N-chlorosuccinimide is rather dissolved not dispersed in the solvent used, preferably completely dissolved, the solvent being selected not to interact with the dissolved N-chlorosuccinimide.

In sub-step 2 the solvent and base conventionally used may be preferably modified and replaced by other compounds not related to known deficiencies, such as heavy metal waste, too heterogeneous reaction procedure etc. Preferred bases are alkali and/or earth alkali hydroxide, more preferably an aqueous solution thereof is used.

In sub-step 3 the known solvent system for the crystallization may be preferably changed to a more optimized alternative. Preferably the solvent system is selected to be ethylacetate/methylcyclohexane.

A further most preferred improvement of the above process is the combination of the improvements of step c) and/or the improvements of step e) and/or the improvements of step f) in order to optimize the synthesis of the produced products. The improvements will be hereinafter explained in detail.

It should be noted that it is within the scope of the present invention that each and every claim of the present application may be understood to refer to each and every other claim, also the resulting embodiments are clearly within the protective scope of the present invention and those skilled in the art understand that the respective embodiments do not leave the scope of the present invention.

### DETAILED DESCRIPTION OF THE INVENTION

Terms not specifically defined herein should be given the meanings that would be given to them by one of skill in the art in light of the disclosure and the context.

For example, the expressions “solution” and “dissolved” or “solved” according to the present invention should be understood in its broadest meaning and include all kind of mixture of solid in a liquid medium such as true solutions, dispersions and the like, unless otherwise stated.

In general, all tautomeric forms and isomeric forms and mixtures, whether individual geometric isomers or optical isomers or racemic or non-racemic mixtures of isomers, of a chemical structure or compound are intended, unless the specific stereochemistry or isomeric form is specifically indicated in the compound name or structure.

Furthermore, it should be noted that the chemical species explicitly mentioned should not be understood to be limited to the specific described species but those skilled in the art know the equivalent compounds having a similar or comparable effect or reaction which should be within the present scope of protection.

For the sake of clarity and in order to provide an overview of the complete multi-step process, all individual steps of the process are described in detail below, although the improvements are particularly realized in process step c) and/or process step e) and/or process step f). The advantages will be explained below. The present invention includes not only the described multi-step process, but also the individual steps of the multi-step process. The entire disclosure of related U.S. Application Publication No. 2006/0025447 A1 is herein incorporated in the present disclosure by reference.

Optimum reaction conditions and reaction times for each individual step may vary depending on the particular reactants employed. Unless otherwise specified, solvents, temperatures and other reaction conditions may be readily selected by one of ordinary skill in the art. Specific procedures are provided in the Synthetic Examples section. Typically, reaction progress may be monitored by high pressure liquid chromatography (HPLC) if desired. Intermediates and products may be purified by crystallization. Unless otherwise described, the starting materials and reagents are either commercially available or may be prepared by one skilled in the art from commercially available materials using methods described in the chemical literature.

Step a)

The starting materials of formula XIV used in this first step are prepared as described by N. Yee, Org. Lett. 2000, 2, 2781-2783, and R. Frutos, Tetrahedron: Asymmetry 2001, 12, 101-104, which are herein incorporated by reference in their entirety. This process is illustrated in Scheme 4.

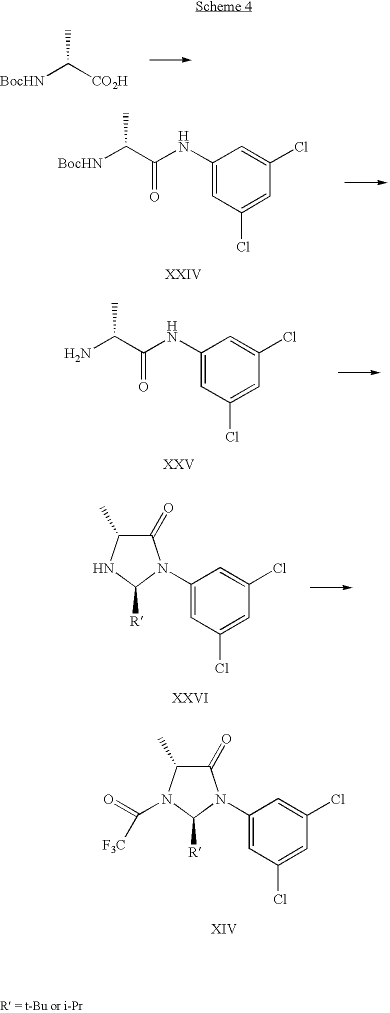

Commercially available D-N-Boc-alanine was reacted with a suitable activating agent, such as isobutyl chloroformate or pivaloylchloride, in the presence of N-methylmorpholine (about −10° C., THF), followed by addition of 3,5-dichloroaniline to give amide XXIV. Deprotection of the crude N-Boc-alaninamide by treatment with TFA in dichloromethane produced amino amide XXV in about 92% yield over two steps. The amino amide was reacted with pivalaldehyde or isobutyraldehyde in refluxing pentane, and the product XXVI was crystallized from the reaction mixture as a single diastereoisomer in >approximately 74% yield. Treatment of XXVI with trifluoroacetic anhydride, in methylene chloride, in presence of triethylamine yielded XIV in about 98% yield.

Step a) of the process of the present invention comprises preferably reacting a compound of formula XIV and a compound of formula XV in the presence of a strong base at a temperature from about 0° C. to about ambient temperature, in an aprotic organic solvent, to provide a compound of formula XVI.

A similar process step is described by N. Yee, Org. Lett. 2000, 2, 2781-2783; R. Frutos, Tetrahedron: Asymmetry 2001, 12, 101-104; U.S. Pat. No. 6,844,360, WO 2004/041827 A2, U.S. Pat. No. 6,852,748 and WO 2004/041273 A1.

The reaction of process step a) of the present invention is preferably performed from about 2° C. to about ambient temperature as compared to about −30 to about 0° C. in the cited references. Examples of compounds of formula XVI (a, b, c, d and e) prepared using this process are illustrated below:

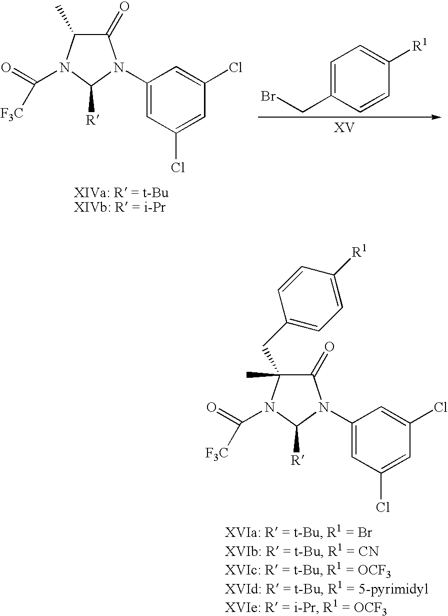

Step a) is performed in an aprotic organic solvent such as THF, ether or dimethoxyethane. Suitable bases preferably include potassium tert-butoxide, lithium diisopropylamide, lithium bis(trimethylsilyl)amide, potassium bis(trimethylsilyl)amide and sodium bis(trimethylsilyl)amide.

Step b)

Step b) of the inventive process comprises deprotection of compounds of formulas XVI. One may accomplish this with a base, optionally in the presence of a phase transfer catalyst such as trimethylbenzylammonium hydroxide, in a suitable solvent such as tetrahydrofuran, 2-methyl tetrahydrofuran or 2-propanol followed by treatment with an acid to form the corresponding amino amide of formula XVII. Specific examples are illustrated below:

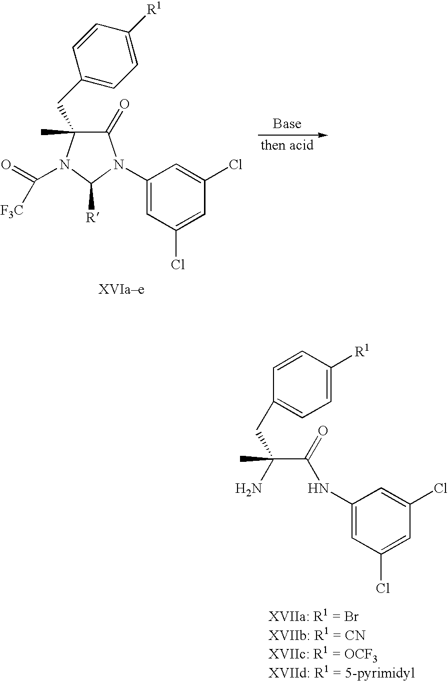

A similar process step is described by N. Yee, Org. Lett. 2000, 2, 2781-2783; R. Frutos, Tetrahedron: Asymmetry 2001, 12, 101-104; U.S. Pat. No. 6,844,360, WO 2004/041827 A2, U.S. Pat. No. 6,852,748, and WO 2004/041273 A1.

Suitable bases for this step preferably include alkali metal hydroxides such as sodium hydroxide or potassium hydroxide. Suitable acids preferably include H₂SO₄ and HCl. Most preferred is potassium hydroxide in isopropyl alcohol followed by 3 M H₂SO₄.

Step c)

Step c) of the inventive process comprises reacting a compound of formula XVII produced in step b) with a compound of formula XVIII, wherein R^{a} is aryl and R^{b} is C₁₋₄ alkyl, and an organic base is used to form a compound of formula XIX, in excellent yield, without the use of a polar organic solvent. Specific examples are shown below:

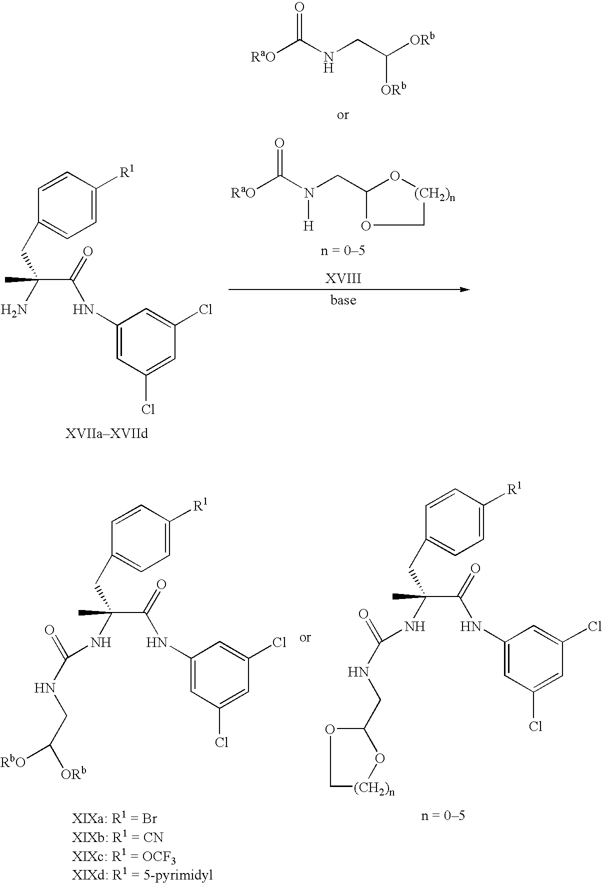

The formation of ureas by reaction of an amine with a phenyl carbamate is documented in the scientific literature (see for example, B. Thavonekham Synthesis, 1997, 1189-1194). Suitable C₁₋₄ alkyl R^{b} groups for the carbamate XVIII in step c) include, for example, methyl, ethyl and cyclobutyl.

Conventionally, step c) is performed in a polar organic solvent, such as dimethylsulfoxide (DMSO). Suitable organic bases conventionally used include, for example, triethylamine, diisopropyl ethylamine, N-methylmorpholine and pyridine.

Using a polar solvent in the preparation of a compound of formula XIX (hereinafter also referred to as “synthesis intermediate 3”) such as dimethylsulfoxide as the solvent for the reaction, leads to a number of disadvantages. For example the solvent must be removed during the further working up of the product, for example with an aqueous extraction. This aqueous extraction requires an additional process step which enhances the complexity of the multi-step process causing superfluous time and effort. In case dimethylsufoxide (DMSO) is used, the contaminated waste water must be decontaminated in a specific way, because that contaminated water may not be supplied to a waste water disposal plant. DMSO is a known toxic organic chemical which has the ability to penetrate human skin and, therefore, must be treated as potent skin penetrator.

According to the present improvement the organic solvent, such as dimethylsulfoxide in step c), is preferably omitted. Therefore, reaction is preferably performed in the presence of a base which simultaneously also serves as a solvent. According to a preferred embodiment of the present invention the base represents a liquid compound. The base may be selected from the group consisting of triethylamine, diisopropyl ethylamine, N-methylmorpholine, pyridine and/or trimethylamine.

Surprisingly it was found that omitting the organic solvent leads to essentially purer products of formula XIX compared to prior art.

A further preferred improvement of step c) may be to perform the crystallization of the product in a solvent system comprising or consisting of alcohol/water. The alcohol may be selected from ethanol, methanol, isopropanol, n-propanol, n-butanol and/or tert.-butanol. Particularly use is made of a methanol/water mixture. The ratio of the mixture of alcohol/water is preferably adjusted in the range from about 3:1 to about 1:3, more preferably about 2:1 to about 1:2, particularly about 1.5:1 to about 1:1.5.

Optionally, the crystallization is performed after previously adjusting the pH value of the product solution with an acid, for example citric acid, tartaric acid, oxalic acid and/or succinic acid to an acidic milieu. Preferably, the pH value may be adjusted in the range from about 4.5 to about 7, particularly preferred in the range from about 5 to about 6.5

It was surprisingly found, that using an alcohol/water system instead of the usually employed solvents such as ethyl acetate/N-heptane mixtures results in products, which may be isolated much faster due to a better filtration ability of the obtained product of formula XIX.

The better filtration ability of the inventive product leads to shorter batch frequencies in operation, which is essential particularly in large-scale operations. The better filtration ability of the product results in an improved after-washing characteristic, which leads to an increased product quality. The optimized crystallisation protocol of the present invention allows for a decrease of the content of by-products, for example, phenol, below the limit of detection. The removal of by-products results to avoid the formation of halogenated by-products in the later halogenation step e) which represent supplementary or additional consumers which prevent the calculation of the exact amount of the halogenation agent used.

According to a most preferred embodiment the general production of step c) is performed as follows:
To a suspension of a compound of general formula XVII and a base such as triethylamine, diisopropyl ethylamine, N-methylmorpholine and/or pyridine a solution of a compound of general formula XVIII is dosed during a suitable period of time such as about 5 to about 100 minutes, preferably about 60 minutes, more preferably about 30 minutes, at a suitable temperature such as about 20° C. to about 100° C., preferably about 50° C. to about 80° C., most preferably about 68° C. to about 72° C. The addition of a compound of formula XVIII may be performed in the presence of a salt such as anhydrous trisodium phosphate, sodium carbonate, potassium carbonate, caesium carbonate, if desired or required. The use of caesium carbonate is not preferred. The reaction mixture is preferably reacted for an appropriate period of time, for example about one or several hrs, at a suitable temperature such as about 20° C. to about 100° C., preferably about 50° C. to about 80° C., more preferably about 68° C. to about 72° C., until the reaction is completed. Optionally the base may be removed preferably by distillation. Thereafter the reaction mixture is cooled and may be worked-up as usual to obtain the product of formula XIX.

Preferably the working-up may be performed in that the residue is taken up or diluted with an alcohol such as methanol, ethanol, isopropanol, n-propanol, n-butanol and/or tert.-butanol, which may be distilled off at suitable temperature/pressure conditions such as about 20° C. to about 100° C./about 100 mbar, preferably about 30° C. to about 70° C./about 100 mbar, more preferably about 40° C. to about 60° C./about 100 mbar, most preferably about 40° C./100 mbar. The resulting residue may be again dissolved in an alcohol such as methanol, ethanol, isopropanol, n-propanol, n-butanol and/or tert.-butanol at an appropriate, preferably elevated, temperature such as about 40° C. to about 70° C., preferably about 50° C. to about 65° C., more preferably about 50° C. Alternatively, water is added after the addition of alcohol at an appropriate, preferably elevated, temperature of particularly above approximately 60° C. Then the product may be either crystallized to isolate the obtained crystals or the reaction solvent may be adjusted to a slightly acid pH value and then crystallized by the addition of a solvent such as water. Thus, the compound of general formula XIX is obtained.

TABLE 1
Stoichiometry of the reaction according to step c) -
Formation of a compound of formula XIX in the presence
of a salt, pH adjustment prior to the addition of water:

| Reagent | Preferred Mol-% ranges |
|---|---|
| Compound of formula XVII | 100 |
| Salt | About 105 to about 150 |
| Base | About 1000 to about 10000; |
| | particularly about 6000 to about 9000 |
| Compound of formula XVIII | About 110 to about 200; |
| | particularly |
| | about 125 to about 180 |

TABLE 2
Stoichiometry of the reaction according to step c) -
Formation of a compound of formula XIX without
a salt and without pH-adjustment

| Reagent | Preferred Mol-% ranges |
|---|---|
| Compound of formula XVII | 100 |
| Base | About 200 to about 600; |
| | particularly |
| | about 300 to about 500 |
| Compound of formula XVIII | About 110 to about 200; |
| | particularly |
| | about 125 to about 180 |

The novel compounds of the following formula XIX produced in this step are another aspect of the present invention: 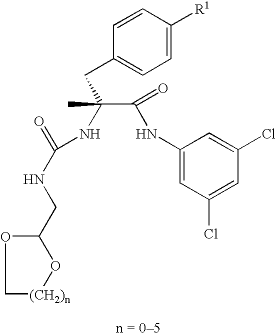
wherein R¹ is selected from bromo, trifluoromethoxy, cyano and pyrimidin-5-yl (or 5-pyrimidyl) optionally mono- or di-subsituted by NH₂.
Step d)

Step d) of the inventive process comprises reacting a compound of formula XIX produced in step c) with a compound of formula (R^{c})₃P, wherein R^{c} is C₁₋₄ alkyl, C₃₋₆ cycloalkyl or aryl, a carbon tetrahalide and a tri-C₁₋₆ alkylamine in an aprotic organic solvent, followed by adding an acid to form a compound of formula XX, in excellent yield. Alternatively, reacting a compound of formula XIX produced in step c) with a compound of formula (R^{c})₃PX₂, wherein R^{c} is C₁₋₄ alkyl, C₃₋₆ cycloalkyl or aryl, X is a halide, and a tri-C₁₋₆ alkylamine in an aprotic organic solvent, followed by adding an acid provides a compound of formula XX. Another alternative is to react a compound of formula XIX produced in step c) with a boronic acid compound ArB(OH)₂, wherein Ar is an aromatic carbocyclic group substituted with one or more electron withdrawing groups, in an aprotic organic solvent to form a compound of formula XX. Specific examples are shown below:

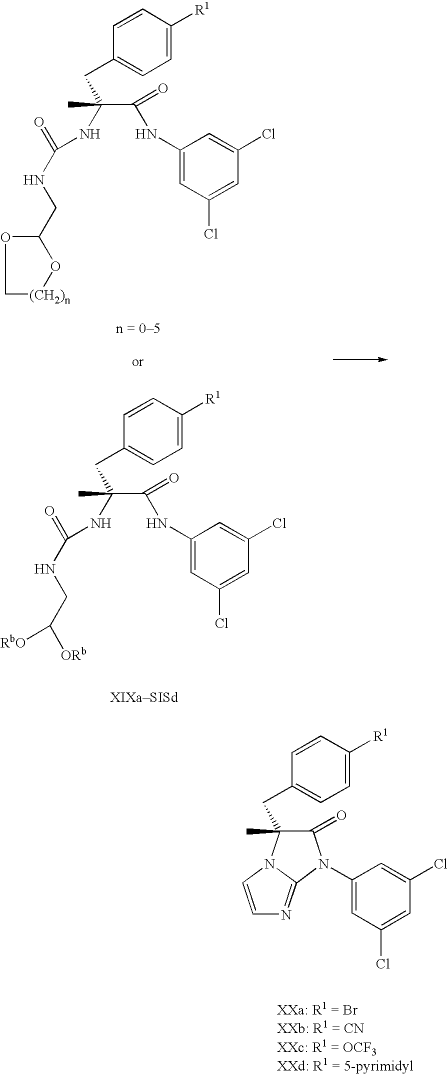

The dehydration of an urea and subsequent cyclization to a guanidine derivative is described in Frutos et al., U.S. Pat. No. 6,414,161. However, in contrast to the procedure described in Frutos et al., in the process of the present invention the intermediate guanidine derivatives are not isolated and undergo a spontaneous cyclization to give the final bicyclic products of formula XX. Furthermore, use of the reagents (R^{c})₃PX₂, for the dehydration/cyclization step is not described in Frutos et al.

A preferred carbon tetrahalide to use in this step is carbon tetrachloride and a preferred tri-C₁₋₆alkylamine is triethylamine.

Step d) is performed in an aprotic organic solvent. Suitable aprotic solvents for performing step d) when reacting XIX with (R^{c})₃P or (R^{c})₃PX₂ include, for example, dichloromethane and acetonitrile. Examples of suitable (R^{c})₃P in step d) include trimethylphosphine, triethylphosphine and triphenylphosphine. Suitable carbon tetrahalides in step d) include, for example, carbon tetrachloride, and carbon tetrabromide.

Examples of suitable (R^{c})₃PX₂ in step d) include triphenylphosphine dichloride and triphenylphosphine dibromide. Examples of suitable acids in step d) include hydrochloric acid and 4-toluenesulfonic acid.

Examples of suitable boronic acid compounds that may be employed for this conversion are compounds of the formula ArB(OH)₂, wherein Ar is an aromatic carbocyclic group, such as a phenyl or naphthyl group, substituted with one or more electron withdrawing groups, such as haloalkyl, halogen and nitro. Specific examples that may be mentioned are compounds 3a to 3d below:

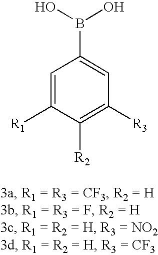

Suitable organic solvents for performing step d) when reacting XIX with the boronic acid compound include relatively high boiling point organic solvents, such as toluene, xylenes and isobutyl acetate.

Step e)

Step e) of the inventive process is a halogenation step that preferably comprises reacting a compound of formula XX produced in step d) with a compound of formula XXI (a compound of formula XXI-1 or alternatively a compound of formula XXI-2 as shown below) wherein Y is halide, in an aprotic organic solvent to form a compound of formula XXII. Specific examples, wherein R¹ is trifluoromethoxy, bromo, cyano and 5-pyrimidyl are shown below:

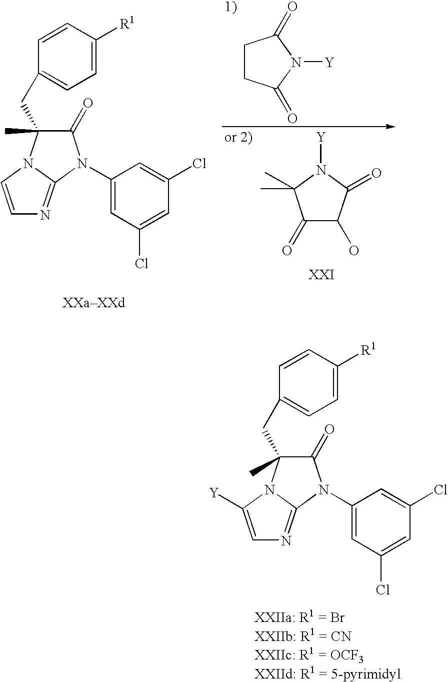

This type of halogenation step is described in U.S. Pat. Nos. 6,492,408, and in 6,844,360, WO 2004/041827 A2, U.S. Pat. No. 6,852,748 and WO 2004/041273 A1.

In one embodiment of the present invention, the Y group in halogenated compounds of formula XXII is preferably bromo and iodo. In a more preferred embodiment, Y is bromo.

If iodination is conducted in step e), it is preferably done in the presence of a Lewis acid such as pyridinium p-toluenesulfonate. The bromination in step e) proceeds most cleanly and in greatest yield if the reaction is run in the presence of a base such as triethylamine, potassium carbonate, N,N-diisopropyl ethylamine, caesium carbonate, sodium carbonate or sodium phosphate, and preferably in dimethoxyethane or isopropyl acetate. The use of caesium carbonate is not preferred.

Step e) can be performed at a wide range of reaction temperatures, but preferably in the range of about −20° C. to about 60° C., more preferably at about −10° C. to about 40° C., more preferably about −5° C. to about 30° C., more preferably about 0° C. to about 25° C.

Conventionally, step e) is performed in an aprotic organic solvent. Suitable aprotic organic solvents include, for example, dichloromethane, acetone, ethylene glycol dimethyl ether, and diglyme.

According to the present invention step e) may be preferably modified in that the compound of formula XXI, serving as halogenation agent, is slightly soluble in the aprotic organic solvent used. The expression “slightly soluble” should be understood in the sense that the compound is not completely solved, but only a little or minor part of the compound may be solved and the rest is not. Therefore, the solubility of the halogenation agent should be selected to be low in the solvent used. More preferably the solubility of the halogenation agent should be selected to be as low as possible in the solvent used.

In a preferred embodiment of the present invention N-bromosuccinimide (NBS), N-iodosuccinimide (NIS) or N,N-dibromodimethylhydantoin are the halogenation agents.

Therefore, the solvent is preferably selected in such a manner that N-bromosuccinimide, N-iodosuccinimide (NIS) or N,N-dibromodimethylhydantoin is only slightly or not dissolved in the solvent used. Therefore, solvents such as dimethoxyethane (DME), diglyme and the like wherein the halogenation agent is completely solved, are not in accordance with the improvement of step e).

Examplarily mentioned solvents, wherein a halogenation agent such as N-bromosuccinimide or N-iodosuccinimide (NIS) or N,N-dibromodimethylhydantoin is only slightly solved, may be isopropyl acetate, ethyl acetate, n-propyl acetate and/or n-butyl acetate. The solvent may contain one solvent alone or a mixture of two or more solvents may be used.

According to the present invention it is avoided to pre-dissolve the halogenation agent in the solvent used during the reaction of step e) in order to avoid a decomposition of the solved halogenation agent in a strong exothermal and uncontrollable process. Additionally, according to the inventive process it is possible to avoid undesired by-products such as halogenated solvent derivatives, which are completely undesired because they reduce the yield, decrease the purity of the product and lead to further undesired reactions.

A further advantage of the inventive process is that the amount of the required halogenation agent such as N-bromosuccinimide (NBS) or N,N-dibromodimethylhydantoin may be reduced which represents benefits in view of economical aspects, better yield, improved purity of product and lower costs.

According to a preferred embodiment process step e) of the present invention is performed in that a compound of formula XX is reacted to a compound of formula XXII, wherein the halogenation agent of formula XXI (XXI-1 or XXI-2) is added as solid in portions to a solution comprising the compound of formula XX and an aprotic organic solvent, which is selected in such a manner that the compound of formula XXI is only slightly or more preferably not soluble in the aprotic organic solvent used.

Furthermore, it is preferred that the halogenation agent is used in solid form. More preferably the halogenation agent being dosed in portions, particularly defined portions, preferably as solid, to the solvent used, i.e. the total amount of halogenation agent is not added at once but it is divided in a number of small amounts and added step by step.

The addition of the halogenation agent in portions shows the benefit that the halogenation agent such as N-bromosuccinimide (NBS) or N-iodosuccinimide (NIS) or N,N-dibromodimethylhydantoin reacts step by step and in a controlled manner with the educt, leading to a more reliable process. Further, the mentioned modified procedure of step e) allows to omit a further process step according to which the halogenation agent must be separately dissolved in a suitable solvent (for example the preparation of a solution of N-bromosuccinimide (NBS) in a solvent such as dimethoxyethane (DME)) in a separate device.

Furthermore, solvents as usually employed such as dimethoxy ethane are known potential alkylation agents which represent potential mutagenic substances, and may be avoided in the process of the invention as far as possible.

The modified process is a simplification of prior art processes: changeovers to different solvents are avoided. Introducing one solvent wherein the halogenation agent is slightly or not solved allows to omit such solvent changeovers. A preferred solvent universally usable is, for example, isopropyl acetate. This procedure leads to an essentially simpler process.

During the development of an improved process it was found that already a lower concentration of water in solvents results in improved results with regard to the halogenating effects, such as the brominizing (better quality and yield). Therefore, it is favourable to control the water content. According to an embodiment of the present invention it is preferred if the presence of compounds having nucleophilic groups such as hydroxyl groups containing compounds, e.g. water or alcohols, or primary and secondary amines and the like are reduced to a minimum. Preferably such nucleophilic compounds are reduced to be equal or less than about 3000 ppm, more preferably equal or less than about 2000 ppm, particularly such compounds should be excluded insofar as possible.

Finally, the preferred use of isopropyl acetate as solvent has a variety of additional preferences. For example the industrial safety is improved: The use of isopropyl acetate in great scale is a further reason to prefer isopropyl acetate vis-à-vis other solvents, because isopropyl acetate neither forms ether peroxides nor it is a mutagenic substance. Furthermore, the preferred use of isopropyl acetate as solvent offers the possibility, if required, to drain the solution effectively by an azeotropic distillation, a possibility that is not readily achieved with other solvents.

According to a most preferred embodiment the general production of step e) is performed as follows:

To a solution of compound of general formula XX preferably a base such as potassium carbonate or triethylamine is added and thereafter the compound of general formula XXI, preferably used in solid form, is charged, preferably in small portions, at a suitable temperature such as about 20° C. to about 25° C. After the layers formed are separated the solvent of the organic phase is removed, preferably by distillation to dryness at suitable temperature/pressure conditions such as about 60° C./about 100 mbar and the residue is worked-up in usual manner.

For working-up, for example, the residue is taken up or diluted in an alcohol such as methanol, ethanol, isopropanol, n-propanol, n-butanol and/or tert.-butanol at a suitable, preferably elevated, temperature. During cooling down of the solution the product crystallizes. The product of general formula XXII may be isolated.

Step f)

Step f) of the inventive process comprises reacting of a compound of formula XXII produced in step e) with a compound of formula R^{d}MgY, wherein R^{d} is C₁₋₆ alkyl or C₃₋₆ cycloalkyl, and Y is halide, sulfur dioxide and N-chlorosuccinimide followed by a base and a compound of formula XXIII in an aprotic organic solvent, to form a compound of formula I without isolation of intermediates formed during this step. Specific examples are illustrated below. R² and R³ are as defined above.

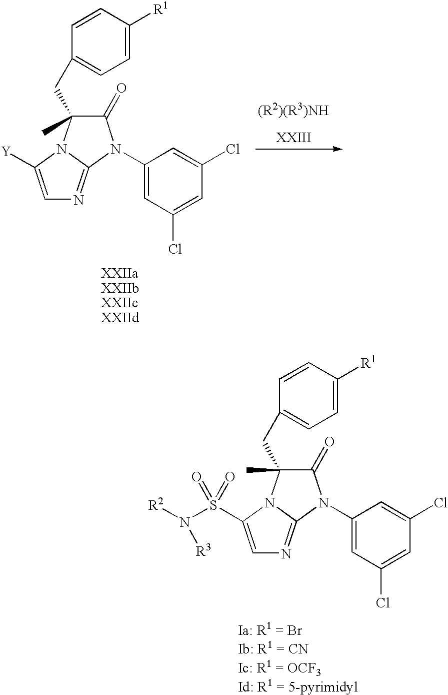

A similar process step is described in U.S. Pat. Nos. 6,492,408, 6,844,360, WO 2004/041827 A2, U.S. Pat. No 6,852,748 and WO 2004/041273 A1. This process step is performed without isolation of any of the intermediates produced during the process. This one-pot process is not disclosed in the above cited reference.

Suitable compounds R^{d}MgY in step f) include, for example, isopropylmagnesium chloride, isopropylmagnesium bromide, cyclopentylmagnesium chloride and cyclopentylmagnesium bromide.

When the R¹ group is 5-pyrimidyl (XXIId, for example) it is necessary to pre-mix an organic base such as N,N,N′,N′-tetramethylethylene diamine, bis[2-(N,N-dimethylamino)ethyl] ether and N,N,N′,N′,N″-pentamethyldiethylenetriamine with R^{d}MgY, prior to reacting with the compound of formula XXIId. This will prevent addition of R^{d}MgY to the 5-pyrimidyl group. This novel process is another aspect of the present invention and is not disclosed in the scientific literature.

The reaction of step f) may be divided in the sub-step 1, which represents the N-chlorosuccinimide oxidation, a reaction which is per se known by those skilled in the art, sub-step 2, which represents the sulfamidation, a reaction which is also per se known by those skilled in the art, and optionally sub-step 3, which represents the crystallization of the crude product of formula I. It should be noted that the N-chlorosuccinimide may be replaced by any other suitable reagent.

The addition of R^{d}MgY and the subsequent addition of sulfur dioxide is preferably performed at a temperature of about −40° C. to about −15° C., preferably about −25° C. to about −15° C. The reaction with N-chlorosuccinimide is preferably conducted at a temperature of about −20° C. to about 10° C., preferably about −15° C. to about 0° C. Addition of a compound of formula XXIII is preferably performed at room temperature.

Conventionally, step f) is carried out in an aprotic organic solvent, preferably tetrahydrofuran. Suitable bases for use in step f) include, for example, triethylamine, diisopropylethylamine, potassium carbonate, caesium carbonate and sodium carbonate. The addition of a compound of formula XXIII is usually and preferably carried out in the presence of water as a co-solvent and even more preferably in the presence of water and dimethylformamide (DMF). It has been found that water accelerates the formation of the product. This step has been performed with up to 10-25% of water in tetrahydrofuran.

However, the above described procedure has a number of deficiencies which are overcome according to the present invention as follows:

Sub-step 1

Sub-step 1 represents the N-chlorosuccinimide oxidation of the sulfinate intermediate, which is described in the experimental section in detail. Conventionally, an aprotic solvent, preferably tetrahydrofuran (THF), is used as suspending solvent of N-chlorosuccinimide as described above, which do not offer satisfying results. Therefore, solvents such as tetrahydrofuran should be completely avoided in sub-step 1.

The improvement according to the present invention is to provide preferably a solvent wherein N-chlorosuccinimide is rather dissolved but not dispersed or suspended in the solvent used, the solvent being selected not to interact with the dissolved N-chlorosuccinimide. Therefore, the inventive solvent of sub-step 1 may be selected from acetonitrile, propionitrile, benzonitrile. For example, if a solvent such as acetonitrile is used, it dissolves N-chlorosuccinimide prior to the sulfinate oxidation. Further the solvent and N-chlorosuccinimide do not hazardously interact in the sense that unfavourable reactions may not occur such as, for example, an undesired thermal runaway decomposition. Such a performance during an operation is a potential hazard for large scale reactions. Moreover, N-chlorosuccinimide is dissolved and no longer suspended, which decreases the heterogeneity of the process. The solvent may contain one solvent alone or a mixture of two or more solvents may be used.

Sub-step 2

Conventionally in process sub-step 2, which represents the sulfamidation in step f), co-solvent dimethylformamide is preferably used. However, dimethylformamide is a known teratogenic compound which is usually left an a remainder in the product obtained.

According to the present invention dimethylformamide is completely avoided and it is preferably used the same solvent as used in sub-step 1, that is acetonitrile, propionitrile, benzonitrile. Therefore, the acetonitrile solvent preferably used for dissolving N-chlorosuccinimide also acts as non-protic co-solvent for the sulfamidation. The most preferred reaction medium in sub-step 2 is a mixture of water/acetonitrile, particularly a mixture of tetrahydrofuran/water/acetonitrile

For sluggishly reacting amine coupling components (primary amines) alkali carbonates should be used as bases. Organic bases lead to hydrolysis of the sulfonylchloride intermediate. Anorganic bases are for example alkali or earth alkali carbonates and from the alkali carbonates caesium carbonate works optimal. However, using caesium carbonate has the disadvantage of heavy metal waste water streams which must be disposed and the product obtained is usually contaminated with heavy metal caesium.

According to the present invention caesium carbonate should be avoided in sub-step 2. Therefore, it is a further preferred improvement that caesium carbonate may be substituted by an alkali and/or earth alkali hydroxide, more preferably by an aqueous solution thereof By online controlling the pH value of the reaction mixture, for example between about 8.0 to about 9.0 for example via slow addition of the alkali and/or earth alkali metal hydroxide solution, the reaction proceeds smoothly and the formation of the hydrolysis side product is suppressed. Moreover the reaction temperature can be increased to a higher temperature such as up to about 40° C. reducing the reaction time needed until full conversion.

Furthermore, the coupling process provides an improved homogeneous reaction. By using an aqueous solution of an alkali and/or earth alkali metal hydroxide and the heterogeneity of the sulfamidation step is reduced to a biphasic reaction mixture almost free of any solid phase. According to a preferred embodiment the compound of formula XXIII is used in sub-step 2 in an aqueous solution which further supports the homogenity of the reaction procedure.

Sub-step 3

Conventional sub-step 3, which represents the crystallization of the obtained product of formula I in crude form, is usually performed from the mixed solvent system of ethylacetate/n-heptane. The results are not satisfying. Therefore, a crystallization system is needed which produces the product of formula I in crude form in high purity. Preferably the impurities should not exceed 0. 1%. Such an improved sub-step 3 would allow for a higher flexibility for the solvent systems to be used in the succeeding crystallization step, which produces the desired polymorph of the pharmaceutically active ingredient used in a pharmaceutical drug product.

As a result of the above, the known solvent system for the crystallization is changed to a more optimized alternative in sub-step 3 according to the present invention. The solvent system for crystallization may be preferably switched to a solvent mixture comprising or consisting of ethylacetate/methylcyclohexane from ethylacetate/n-heptane leading to a product with no impurity above approximately 0.1%, a finding which is totally unexpected. However, the yield reduced by approximately 5 to 10% (increased mother liquor losses) which may be readily accepted due to the significantly improved quality.

According to a most preferred embodiment the general production of step f) is performed as follows:
To a solution of a compound of formula XXII is added a compound R^{d}MgY, wherein R^{d} is C₁₋₆ alkyl or C₃₋₆ cycloalkyl, and Y is halide, at low temperatures such as about −30° C. to about −20° C. After completion of the reaction a solution of sulphur dioxide is added to the reaction mixture at a suitable temperature such as about −100° C. to about +10° C., preferably about −60° C. to about +5° C., more preferably about −40° C. to about 0° C., particularly about −30° C. to about −20° C. The resulting solution is added to a cold solution of N-chlorosuccinimide in a solvent, preferably in acetonitrile, keeping the internal temperature very low, for example, below about 0° C. Then, a solution of a compound of formula XXIII is added at a suitable temperature such as about 0° C. to about 15° C. to the reaction mixture. Hereafter, the reaction mixture is heated, preferably immediately heated, usual temperature ranges may be about 10° C. to about 100° C., preferably about 20° C. to about 80° C., more preferably about 30° C. to about 60° C., particularly about 35° C. to about 40° C. An aqueous solution of an alkali or earth alkali metal such as NaOH, KOH, LiOH, Ca(OH)₂ and/or Mg(OH)₂, for example a 50% aqueous solution of one or more alkali and/or earth alkali hydroxides, is dosed, preferably continuously dosed, in the reaction mixture, to maintain the reaction pH preferably between about 7 to about 10, more preferably about 8 and about 9, most preferably about 8.2 to about 8.7. After the reaction is finished the solvent may be removed. The working-up is done in an usual manner.

Preferably the working-up is performed in that the residue is partitioned between two solvent such as ethyl acetate and water. After adjusting the pH of the aqueous phase preferably in the range from about 5 to about 7 by adding an acid such as concentrated hydrochloric acid, the phases may be separated. After washing the organic layer in usual manner the product solution may be dried. The product of formula I is obtained in crude form.

TABLE 3
reaction stoichiometries for a compound of formula I in crude form

| Reaction | Preferred mol % ranges |
|---|---|
| Compound of formula | 100 |
| XXII | |
| R^{d}MgY | About 100 to about 130, particularly about 110 to |
| | about 120 |
| Sulfur dioxide | About 100 to about 150, particularly about 115 to |
| | about 140 |
| N-chlorosuccinimide | About 100 to about 170, particularly about 120 to |
| | about 160 |
| Compound of formula | About 100 to about 200, particularly about 130 to |
| XXIII | about 180 |

The crude product of formula I may be preferably crystallized with the solvent system comprising ethylacetate/methylcyclohexane to obtain pure product of formula I.

### PREFERRED EMBODIMENTS OF COMPOUNDS OF FORMULA (I)

The compounds that may be prepared by the processes of the present invention are compounds of formula I as previously set forth, i.e. compounds of the following formula: 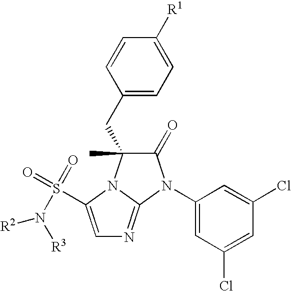
wherein:
R¹ is selected from bromo, trifluoromethoxy, cyano and pyrimidin-5-yl optionally mono- or disubsituted by NH₂ ; and
R² and R³ are each independently selected from the group consisting of
   a) hydrogen; and
   b) a C₁₋₄ straight or branched alkyl group, optionally mono- or disubstituted with moieties independently selected from oxo, —OH, NH₂ and —C(O)NR⁴R⁵, wherein R⁴ and R⁵ are independently selected from:
      (1) hydrogen, and
      (2) a C₁₋₄ straight or branched alkyl group which alkyl group is mono- or disubstituted with moieties independently selected from CONH₂ and OH;
         or
R² and R³, combined with the nitrogen they are bonded to, form:
   (1) a pyrrolidine or piperidine ring, each optionally substituted with the group —C(O)NR⁶R⁷, wherein R⁶ and R⁷ are independently selected from
      a) hydrogen; and
      b) a C₁₋₄ straight or branched alkyl group, optionally mono- or disubstituted with moieties independently selected from oxo, —OH and NH₂;
   (2) a morpholine ring; or
   (3) a piperazine ring;
      or a pharmaceutically acceptable salt thereof.

In another embodiment of the compound of formula I:
R¹ is selected from bromo, trifluoromethoxy, cyano and pyrimidin-5-yl;
R² is H; and
R³ is —CH(R⁸)C(O)NH₂, wherein R⁸ is a straight or branched C¹⁻³ alkyl group; or
R² and R³, together with the nitrogen they are bonded to form a moiety selected from 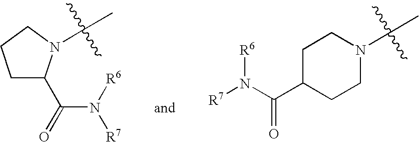
   wherein R⁶ and R⁷ are independently selected from H and straight or branched C¹⁻⁴ alkyl optionally substituted with OH.

Specific examples of compounds of formula (I) that may be prepared using the process of the present invention are the following:

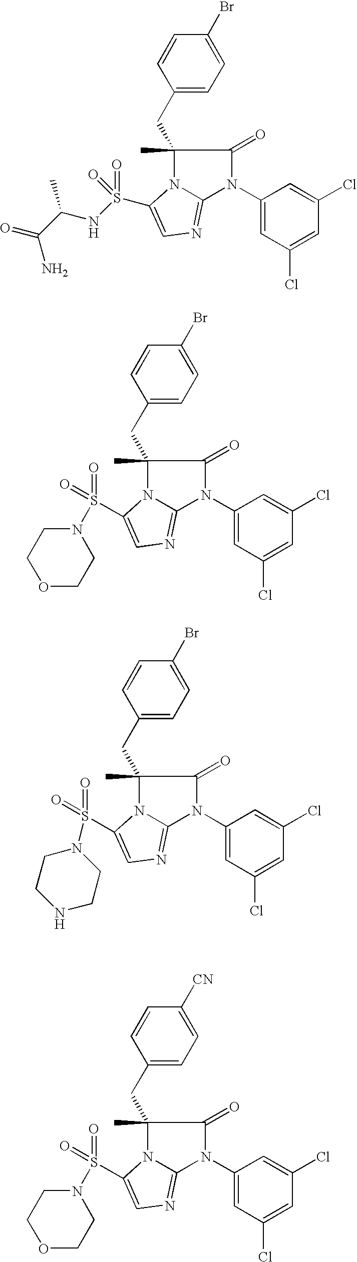

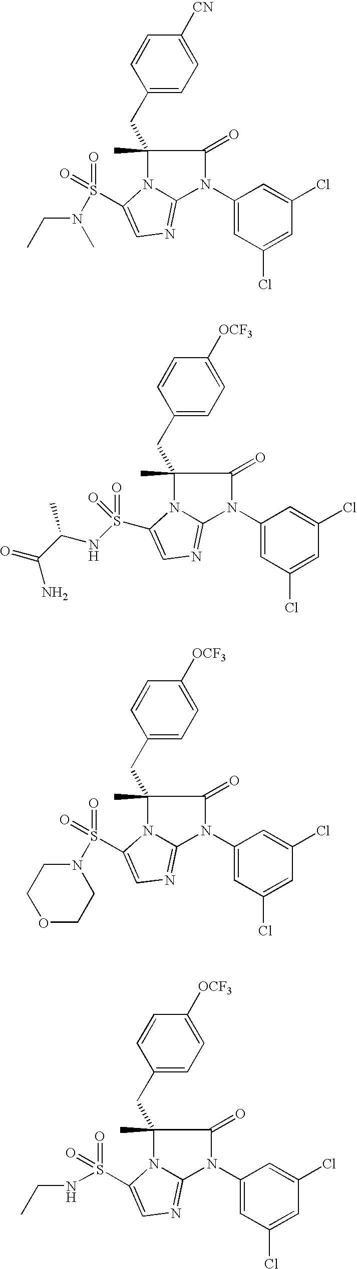

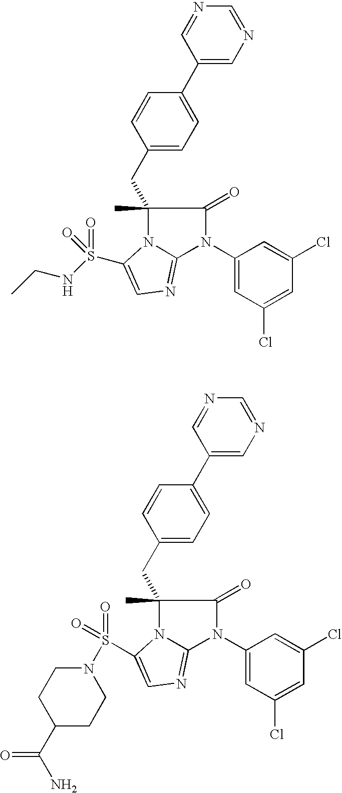

In order that this invention be more fully understood, the following examples are set forth. These examples are for the purpose of illustrating embodiments of this invention, and are not to be construed as limiting the scope of the invention in any way.

### SYNTHETIC EXAMPLES

The following are representative examples that illustrate the process of the present invention.

In the following examples R¹ may be selected from bromo, trifluoromethoxy, cyano, pyrimidin-5-yl or mono- or disubsituted NH₂ and
R² and R³ are as defined above.
   Synthesis of Intermediate 1

### Example 1

Example 1 is directed to the reaction of a compound of formula XIV via a compound of formula XVI (process step a)) to a compound of formula XVII (process step b); synthesis intermediate 1) as follows:

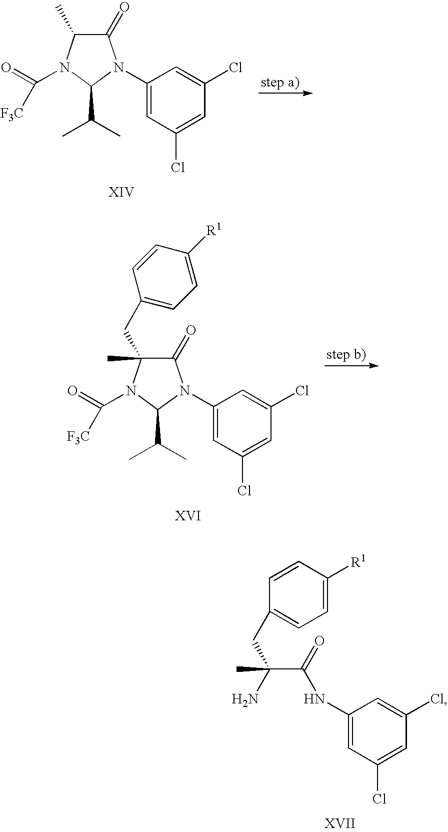

To a solution of a compound of formula XIV (130.0 mmol) and 4-R¹-benzyl bromide (133.0 mmol) in tetrahydrofuran (THF) is added lithium bis(trimethylsilyl)amide (1.0 M solution in THF, 136.5 mmol) at about 0° C. over about 20 min keeping the internal temperature below about 0° C. The resulting mixture is stirred for about 30 min. 10% aqueous ammonium chloride and EtOAc are added. The layers are separated and the organic layer is concentrated to dryness. To the residue 2-propanol and potassium hydroxide (176 mmol) are added and the mixture is heated to about 50° C. for about 4 h. 3 M H₂SO₄ is then added and the mixture is heated to about 70° C. for about 2 h. 2-Propanol is distilled and isopropyl acetate is added. The organic solution is washed with 2 N NaOH and water and then concentrated to dryness. Acetonitrile is added to the residue followed by 4-toluenesulfonic acid monohydrate (136.5 mmol). The mixture is stirred at room temperature for about 10 h. The compound of formula XVII is collected by filtration.

Synthesis of Intermediates 3, 4 and 5

The following scheme 5 shows an overview of the synthesis as described in the following Examples. 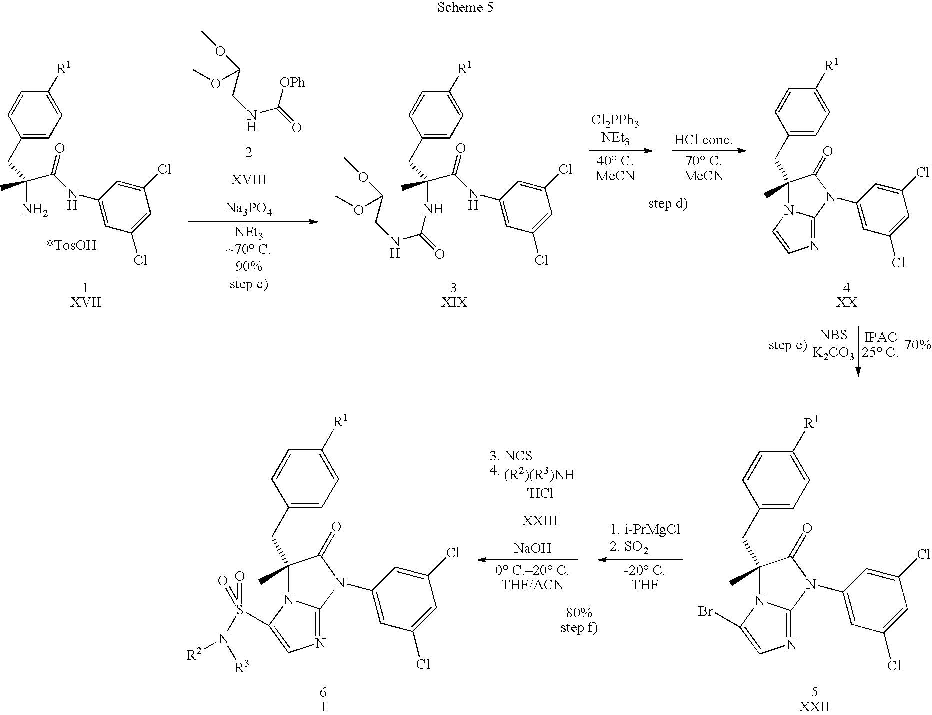
Synthesis of Intermediate 3

In the following Examples 2 to 10 the synthesis according to step c) of the present invention is described.

### Example 2

Formation of an urea compound of formula XIX (synthesis intermediate 3) by reacting the compound of formula XVII with a compound of formula XVIII to form a compound of formula XIX in the presence of sodium phosphate, pH-adjustment to a value of 5 prior to addition of water

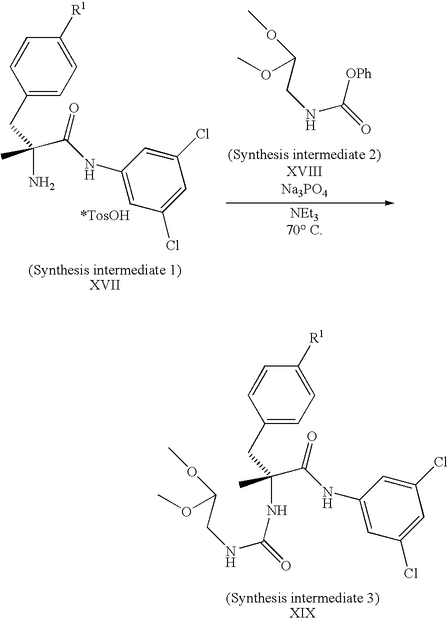

To a suspension of a compound of formula XVII (131 mmol) and anhydrous trisodium phosphate (144 mmol) in triethylamine (1.5 mol) a solution (50% in methylen-tert.-butylether) of a compound of formula XVIII (197 mmol) is dosed during about 5 to about 100 minutes, preferably about 60 minutes, at a temperature of about 20° C. to about 100° C., preferably about 50° C. to about 80° C., most preferably about 68° C. to about 72° C. The reaction mixture is stirred under condensation of the gaseous phase for further about 4.5 hrs at a temperature of about 20° C. to about 100° C., preferably about 50° C. to about 80° C., most preferably about 68° C. to about 72° C., until the reaction is completed (HPLC-analysis: educt<0.5 area-%). Thereafter it is cooled to a temperature of about −10° C. to about 20° C., preferably about −5° C. to about 15° C., more preferably about 5° C. to about 10° C. whereby ethyl acetate is added during an inner temperature of about 50° C. is maintained. After stirring for about 15 minutes, the suspension is sucked off at a temperature of about −10° C. to about 20° C., preferably about −5° C. to about 15° C., more preferably about 5° C. to about 10° C., and the remaining residue is washed with ethyl acetate. The filtrate is distilled off until an oily residue (at about 20° C. to about 100° C./about 100 mbar, preferably about 30° C. to about 70° C./about 100 mbar, more preferably about 40° C. to about 60° C./about 100 mbar, most preferably about 40° C./about 100 mbar) is obtained. After taking up the residue in methanol it is again distilled off at about 20° C. to about 100° C./about 100 mbar, preferably about 30° C. to about 70° C./about 100 mbar, more preferably about 40° C. to about 60° C./about 100 mbar, most preferably about 40° C./about 100 mbar, and the oil is again dissolved in methanol at about 40° C. to about 70° C., preferably about 50° C. to about 65° C., more preferably about 50° C. After the addition of water the reaction solvent is adjusted to a pH value of approximately 5 (pH-paper) with 30% methanolic citric acid and is crystallized by the addition of water in portions (for example about 39 g at about 56° C.; about 205 g at about 35° C. to about 40° C.; about 69 g at about 20° C.) and cooled to about −10° C. to about 20° C., preferably about −5° C. to about 15° C., more preferably about 10° C. The crystals are isolated and subjected to an after-washing with a mixture of methanol/water (1/1) cooled to a temperature of about −10° C. to about 20° C., preferably about −5° C. to about 15° C., more preferably about 0° C. After drying overnight in a vacuum drying cupboard at about 20° C. to about 100° C., preferably about 30° C. to about 90° C., more preferably about 40° C. to about 80° C., most preferably about 50° C. to about 60° C., particularly about 50° C. the compound of formula XIX is obtained.

### Example 3

Formation of an urea compound of formula XIX (synthesis intermediate 3) in the presence of sodium phosphate, pH adjustment to approximately 4 prior to the addition of water.

According to example 2 a crude solution of compound XIX in ethyl acetate is worked up after filtration and crystallized at a pH-value of approximately 4 with a mixture of methanol/water.

### Example 4

Formation of an urea compound of formula XIX (synthesis intermediate 3) in the presence of sodium phosphate, pH adjustment to approximately 6 prior to the addition of water.

According to example 2 a crude solution of a compound of formula XIX in ethyl acetate is worked up after filtration and crystallized at a pH-value of approximately 6 with a mixture of methanol/water.

### Example 5

Formation of an urea compound of formula XIX (synthesis intermediate 3) in the presence of sodium phosphate, pH adjustment to approximately 5.5 prior to the addition of water.

According to example 2 a crude solution of a compound of formula XIX in ethyl acetate is worked up after filtration and crystallized at a pH-value of approximately 5.5 with a mixture of methanol/water.

### Example 6

Formation of an urea compound of formula XIX (synthesis intermediate 3) in the presence of sodium phosphate, pH adjustment to approximately 6.5 prior to the addition of water.

According to example 2 a crude solution of a compound of formula XIX in ethyl acetate is worked up after filtration and crystallized at a pH-value of approximately 6.5 with a mixture of methanol/water.

### Example 7

Formation of an urea compound of formula XIX (synthesis intermediate 3) in the presence of sodium phosphate, pH adjustment to approximately 7 prior to the addition of water.

According to example 2 a crude solution of a compound of formula XIX in ethyl acetate is worked up after filtration and crystallized at a pH-value of approximately 7 with a mixture of methanol/water.

### Example 8

Formation of an urea compound of formula XIX (synthesis intermediate 3) in the presence of sodium phosphate, pH adjustment to approximately 9 prior to the addition of water.

According to example 2 a crude solution of a compound of formula XIX in ethyl acetate is worked up after filtration and crystallized at a pH-value of approximately 9 with a mixture of methanol/water (a methanolic citric acid solution was not added).

### Example 9

Formation of an urea compound of formula XIX (synthesis intermediate 3) without sodium phosphate and without pH-adjustment. Complete precipitation at a temperature of about 60° C. to about 68° C.

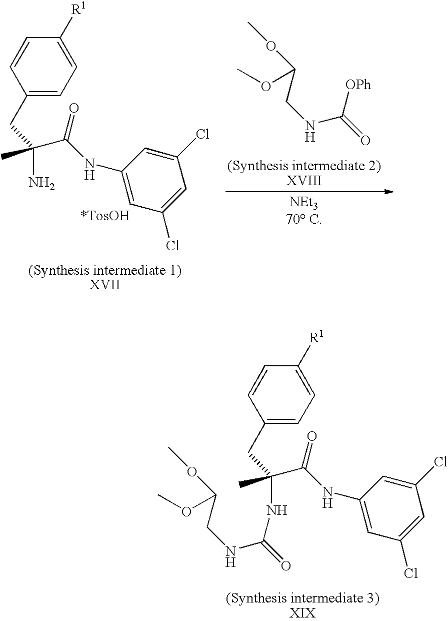

To a suspension of a compound of formula XVII (35 mmol) in triethylamine (144 mmol) a solution (25% in methylene-t-butylene ether) of a compound of formula XVIII (52 mmol) is dosed during about 5 to about 100 minutes, preferably about 30 minutes, at a temperature of about 68° C. to about 72° C. The reaction mixture is stirred under condensation of the gaseous phase for further about 6 hrs at a temperature of about 20° C. to about 100° C., preferably about 50° C. to about 80° C., more preferably about 68° C. to about 72° C., until the reaction is completed (HPLC analysis: educt<0.5 area-%). Optionally the triethylamine is removed by distillation.

Thereafter it is cooled to a temperature of about 20° C. to about 100° C., preferably about 50° C. to about 80° C., more preferably about 68° C. to about 72° C., the reaction mixture is diluted with methanol and it is heated to >about 60° C. While the mixture is still warm (about 60° C. to about 68° C.) water is added and the product is crystallized. After complete addition of water it is stirred at about 68° C. for about 10 minutes and subsequently cooled to about −20° C. to about 20° C., preferably about −10° C. to about 10° C., more preferably about 0° C. After continued stirring for about 10 minutes the crystals are isolated and subjected to an after-washing with a mixture of methanol/water (about 1/1) cooled to about 0° C. After drying overnight in a vacuum drying cupboard at about 20° C. to about 100° C., preferably about 30° C. to about 90° C., more preferably about 40° C. to about 80° C., most preferably about 50° C. to about 60° C., especially about 50° C. The compound of formula XIX is obtained.

### Example 10

Formation of an urea compound of formula XIX (synthesis intermediate 3) with sodium phosphate in DMSO and crystallization from ethylacetate/n-heptane

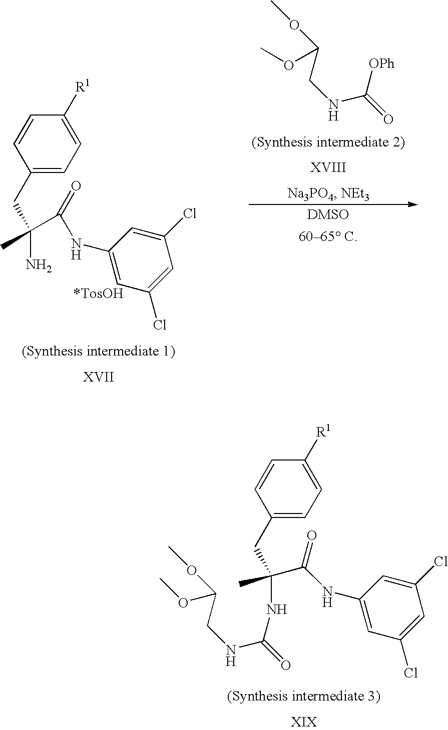

To a solution of a compound of formula XVII (35 mmol) in dimethylsulphoxide triethylamine (21 mmol), anhydrous sodium phosphate (52 mmol) and a compound of formula XVIII (42% in methyl-tert.-butyl ether) (52 mmol) are added at about 20° C. to about 25° C. The reaction mixture is heated to a temperature of about 60° C. to about 65° C. and is stirred under condensation of the gaseous phase at about 60° C. to about 65° C. for about 6 hrs until the reaction is completed (HPLC analysis: educt<0.5 area-%). Thereafter it is cooled to about 20° C. to about 25° C. and ethylacetate and 2.3% sodium carbonate solution is added to the reaction mixture. After separation of the phases, the organic phase is washed with 3% sodium chloride solution and evaporated to half the amount at about 45° C./100 mbar. Subsequently n-heptane (110 ml) is added during about 30 minutes at about 45° C. and the obtained high viscous suspension is cooled to about 10° C. The product obtained after filtration is washed with a mixture of ethylacetate/n-heptane (about 10/1). After drying overnight in a vacuum drying cupboard at about 50° C. the compound of formula XIX is obtained.

### Synthesis of Intermediate 4

### Example 11

Example 11 is directed to the reaction of a compound of formula XIX (synthesis intermediate 3) to a compound of formula XX (synthesis intermediate 4) (process step d) via cyclization of an urea compound (synthesis intermediate 3) with triphenylphosphine dichloride followed by treatment with conc. hydrochloric acid.

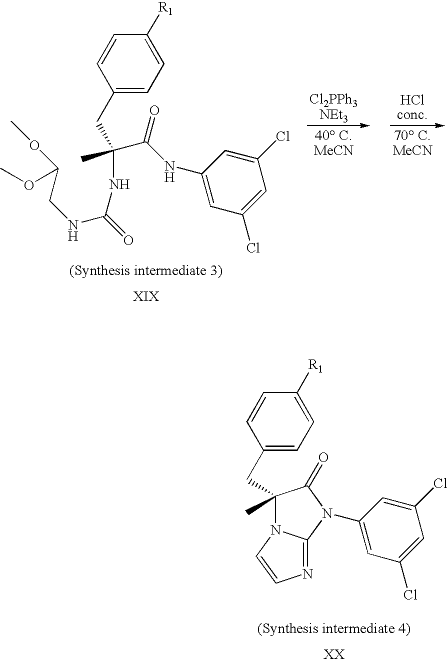

To a suspension of a compound of formula XIX (149 mmol) in acetonitril and triethylamine (594 mmol) a solution of triphenylphosphine dichloride (223 mmol) in acetonitrile is added at about 40° C. to about 45° C. within about 2 hours. The reaction mixture is then heated to about 54° C. and conc. hydrochloric acid (297 mmol) is added, leading to a temperature rise to about 65° C. to about 70° C. The reaction mixture is further stirred at about 70° C. until complete conversion (HPLC analysis) is observed. The reaction mixture is worked up by removing acetonitrile at about 60° C./about 100 mbar and isopropyl acetate is added. The resulting organic phase is washed with water, 10% sodium chloride solution, 5% sodiumhydrogencarbonate solution and 2.5% sodium chloride solution. Isopropyl acetate is removed by distillation and the product solution is cooled to about −16° C. to precipitate triphenylphosphine oxide. The suspension is filtered and the isolated solid is washed with isopropyl acetate. The filtrates are combined, distilled to dryness and redissolved in isopropyl acetate to yield a crude solution of a compound of formula XX (synthesis intermediate 4) which may be directly used for the succeeding bromination step (see preparation of synthesis intermediate 5).

### Synthesis of Intermediate 5

### Example 12

Example 12 is directed to the reaction of a compound of formula XX to a compound of formula XXII (process step e)) and represents in the present case the bromination of an imidazole (synthesis intermediate 4) with N,N-dibromodimethylhydantoin in isopropyl acetate.

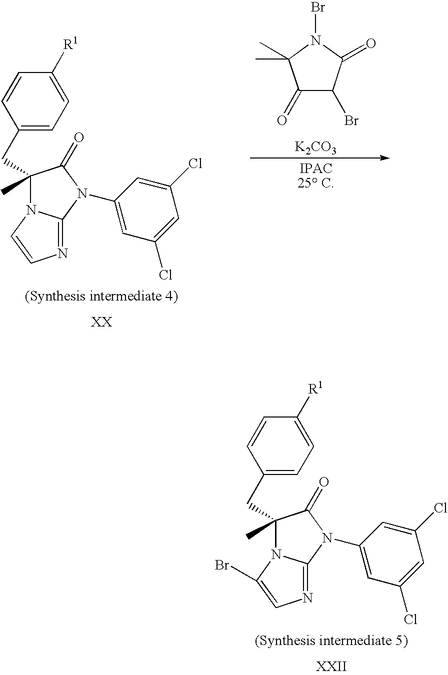

An isopropyl acetate solution of a compound of formula XX (50 mmol based on 100% yield from a compound of formula XIX) is diluted with isopropyl acetate. After addition of potassium carbonate (10 mmol) N,N-dibromodimethylhydantoin (26 mmol) is charged in small portions at about 20° C. to about 25° C. 10% sodium chloride solution is added to the reaction mixture and the layers are separated. The organic phase is distilled to dryness at about 60° C./about 100 mbar and the oily residue is redissolved in n-butanol at about 55° C. to about 60° C. During cooling down of the n-butanol solution to about 20° C. to about 25° C. the product crystallizes. The crystallization is completed by adding isopropanol and water and fuirther cooling to about 0° C. to about 5° C. The product is isolated by filtration and washed with isopopanol/water (v/v=4:1). After drying in vacuum overnight at about 45° C. to about 50° C. the compound of formula XXII (synthesis intermediate 5) is obtained.

### Example 13

Example 13 is directed to the reaction of a compound of formula XX to a compound of formula XXII (process step e)) and represents in the present case the bromination of an imidazole (synthesis intermediate 4) with N-bromosuccinimide in isopropyl acetate.

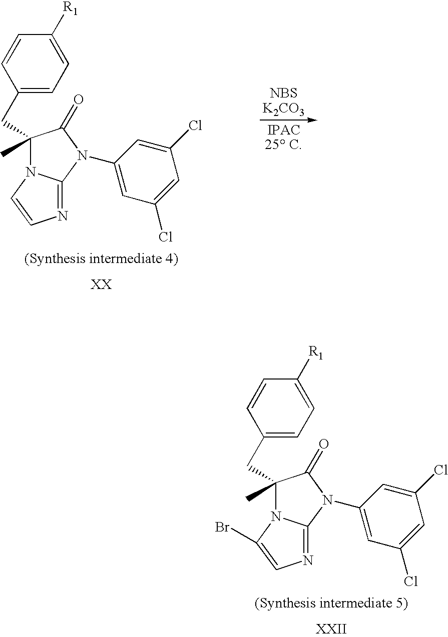

An isopropyl acetate solution of a compound of formula XX (50 mmol based on 100% yield from a compound of formula XIX) is diluted with isopropyl acetate. After addition of potassium carbonate (10 mmol) N-bromosuccinimide (50 mmol) is charged in small portions at about 20° C. to about 30° C., preferably about 20° C. to about 25° C. 10% sodium chloride solution is added to the reaction mixture and the layers are separated. The organic phase is distilled to dryness at about 60° C./about 100 mbar and the oily residue is redissolved in n-butanol at about 55° C. to about 60° C. During cooling down of the n-butanol solution to about 20° C. to about 25° C. the product crystallizes. The crystallization is completed by adding isopropanol and water and further cooling to about 0° C. to about 5° C. The product is isolated by filtration and washed with isopopanol/water (v/v=4:1). After drying in vacuum overnight at about 45° C. to about 50° C. the compound of formula XXII (synthesis intermediate 5) is obtained.

Alternatively, work up is done by adding 5% (w/w) solution of sodium sulfite, separation of the phases, the organic phase is washed with sodium bicarbonate (5% w/w), then it is distilled and the residue is crystallized from n-butanol as described above.

Synthesis of Product I

The following examples 14 and 15 show the formation of a compound of formula I by reacting a compound of formula XXII with a compound of formula R^{d}MgY, wherein R^{d} is C₁₋₆ alkyl or C₃₋₆ cycloalkyl, and Y is halide, sulfur dioxide and N-chlorosuccinimide followed by a base and a compound of formula XXIII:

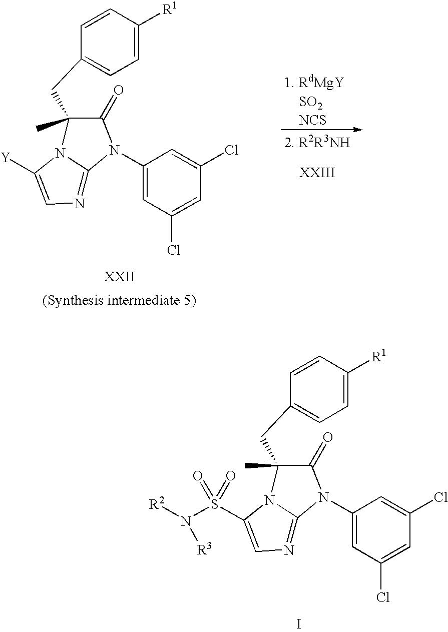

The following scheme 5 shows a detailed overview of the synthesis as described in the following Examples 14 and 15 according to step f).

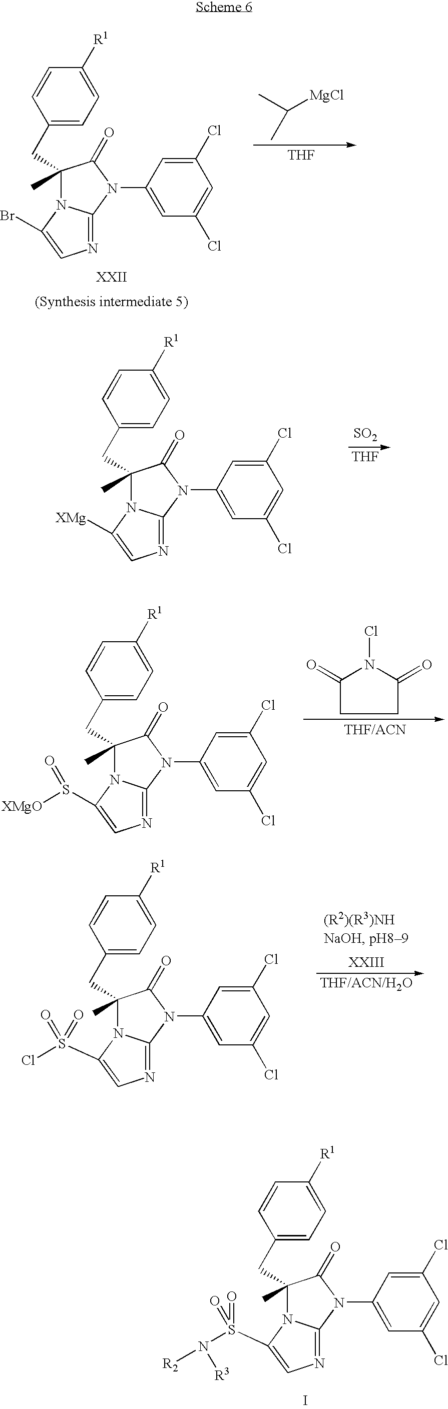

### Example 14

Synthesis of a Compound of Formula I in Crude Form

Acetonitrile/NaOH Coupling at About 40° C. and Ethylacetate/Methylcyclohexane Crystallization

To a solution of a compound of formula XXII (168 mmol) in tetrahydrofuran (THF) or methyl-THF is added isopropylmagnesiumchlorid (20% in THF, 194 mmol) at about −30° C. to about −20° C. After completion of reaction (HPLC control) a 20.1% solution of sulfur dioxide in anhydrous THF (203 mmol) is added to the reaction mixture at a temperature of about −30° C. to about −20° C. The resulting solution is transferred to a second reactor containing an about −10° C. to about 0° C. cold solution of N-chlorosuccinimide (235 mmol) in acetonitrile keeping the internal temperature below about 0° C. After rinsing the transfer equipment with THF a compound of formula XXIII (252 mmol) dissolved in water is added at about 0° C. to about 15° C. to the reaction mixture. Hereafter, the reaction mixture is immediately heated to about 35° C. to about 40° C., while continuously dosing a 50% aqueous solution of sodium hydroxide to maintain the reaction pH between about 8 and about 9. After stirring for additional about 3 to about 4 hours, the period of time depends from the added amount of compound of formula XXIII and the reaction temperature, the reaction is finished (HPLC control) and the solvent is completely removed by vacuum distillation at about 70° C./about 200 mbar. The residue is partitioned between ethyl acetate and water. After adjusting the pH of the aqueous phase to about 5 to about 7 by adding concentrated hydrochloric acid, the phases are separated. The organic layer is further washed with 10% aqueous potassium carbonate solution, diluted hydrochloric acid (0.5 N) and 2.5% aqueous sodium chloride solution. The product solution is azeotropically dried by removing ethylacetate and treated with charcoal for about 10 minutes at about 60° C. After charcoal filtration, which may be optionally performed, the product solution is concentrated. As an alternative it is possible to perform a distillation to dryness and redissolve in ethylacetate. Then methylcyclohexane is added at about 50° C. to about 60° C. After seeding at about 50° C. crystallization occurs and a second portion of methylcyclohexane is added over about 2 hours. The product suspension is cooled about −10° C. to about −20° C. within about 2 hours and stirred at that temperature for about 1 hour. The product is isolated by filtration and washed with ethylacetate/methylcyclohexane (v/v=1:6). After drying in vacuum overnight at about 45 to about 50° C. the product of formula I is obtained.

Crystallization system for a compound of formula I in crude form according to an exemplary system:

Ratio: (bromide starting material):(co-solvent ethylacetate):

| | |
|---|---|
| Actual: | 168 mmol:272.0 ml |
| = | 1 mmol:1.6 ml |
| Alternatives: | 1 mmol:0.5-2.7 ml; preferably 1.0-2.1 ml |

Ratio: (co-solvent ethylacetate):(and solvent methylcyclohexane)

| | |
|---|---|
| Actual: | 272.0 ml:(504.0 + 588.0 = 1092) |
| = | 1 ml:4 ml |
| Alternatives: | 1 ml:2-10 ml, 3-8 ml, 4-6 ml |

### Example 15

Synthesis of a Compound of Formula I in Crude Form

Formation of a compound of formula I by reacting a compound of formula XXII with a compound of formula R^{d}MgY, wherein R^{d} is C₁₋₆ alkyl or C₃₋₆ cycloalkyl, and Y is halide, sulfur dioxide and N-chlorosuccinimide followed by a base and a compound of formula XXIII (original procedure of U.S. patent application Ser. No. 11/188,377 with Cs₂CO₃ coupling in DMF/water/THF at about 20° C. and crystallization from ethylacetate/n-heptane).

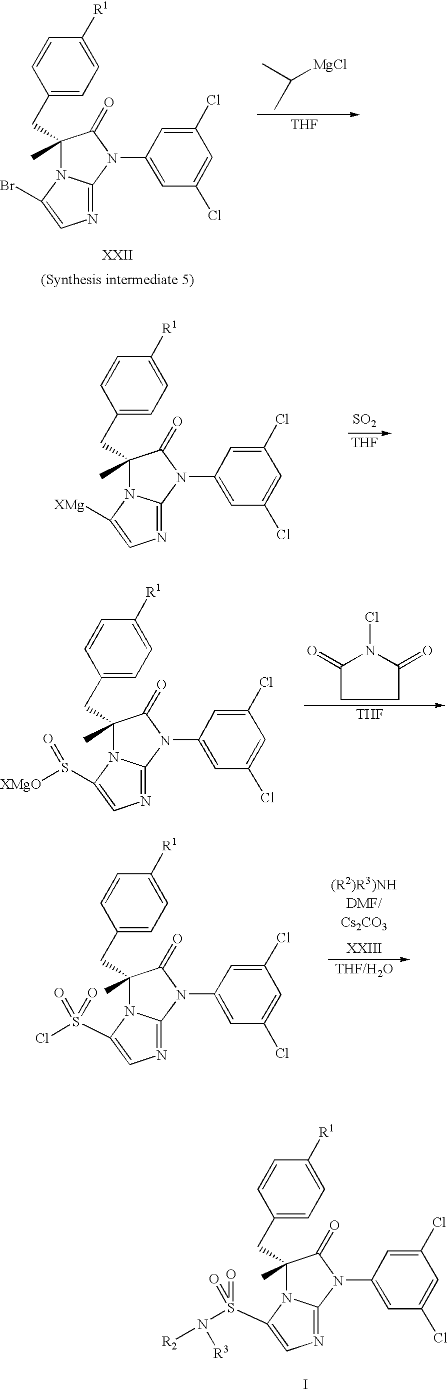

To a solution of a compound of formula XXII (37 mmol) in THF is added isopropylmagnesiumchlorid (2 mol/L in THF, 40 mmol) at about −17° C. to about −22° C. After completion of reaction (HPLC control) a 20.5% solution of sulfur dioxide in anhydrous THF (44 mmol) is added at about −20° C. and the resulting solution is transferred to a second reactor containing an about −5° C. cold solution of N-chlorosuccinimide (52 mmol) in anhydrous THF keeping the internal temperature below about 0° C. After stirring for about 1 hour a compound of formula XXIII (75 mmol) and caesium carbonate (67 mmol) are added at about 10° C., followed by water and N,N-dimethylformamide at the same temperature under vigorous stirring. The hetergeneous reaction mixture is allowed to warm to about 20° C. to about 25° C. and stirring is continued (approx. 20 hours) until HPLC control indicates complete conversion. Water and ethylacetate are added, the phases are separated and the organic phase is evaporated to dryness at about 60° C./about 160 mbar. The residue is redissolved in ethyl acetate and the resulting product solution is washed with 10% aqueous potassium carbonate solution, diluted hydrochloric acid (0.5 N) and 2.5% aqueous sodium chloride solution. The solvent is removed by vaccum distillation at about 60° C./about 100 mbar and the residue redissolved in ethylacetate at about 65° C. n-heptane is added at about 65° C. to about 67° C. and the crystal suspension formed is cooled to about −5° C. to about −15° C. The product is isolated by filtration and washed with ethylacetate/n-heptane (v/v=1:6). After drying in vacuum overnight at about 35° C. the compound of formula I is obtained.

TABLE 4
Purity of I crude batches prepared according to examples 14 and 15:

| | | Impurities with |
|---|---|---|
| Example | HPLC purity (area %) | >0.1 HPLC area % |
| #14 | 99.7 | 0 |
| #15 | 98.8 | 2 |

### Example 16

The compounds of formula I listed below are an illustrative selection prepared by the inventive process using an appropriate imidazolone compound, such as an iodoimidazolone or bromoimidazolone intermediate of formula XXII wherein Y is either I or Br:

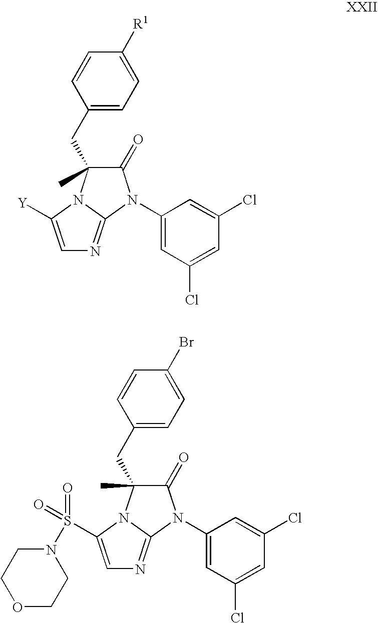

¹H NMR (500 MHz, CDCl₃) δ 7.41 (d, J=1.8 Hz, 2H, ArH), 7.34 (s, 1H, imidazole-H), 7.28 (t, J=1.8 Hz, 1H, ArH), 7.23 (ABq, J=8.4 Hz, 2H, ArH), 6.79 (ABq, J=8.4 Hz, 2H, ArH), 3.78 (m, 5H), 3.21 (m, 5H), 1.95 (s, 3H, CH₃), ¹³C NMR (500 MHz, CDCl₃) δ 172.0, 147.2, 134.1, 133.5, 131.7, 130.1, 129.8, 128.9, 126.0, 121.1, 120.3, 119.2, 69.7, 45.9, 44.8, 42.3, 22.2. MS: m/z 600 (M⁺).

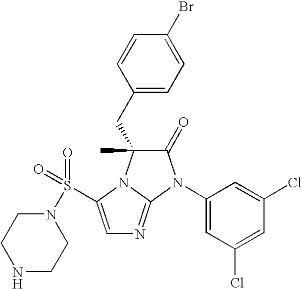

¹H NMR (500 MHz, CDCl₃) δ 7.41 (d, J=1.8 Hz, 2H, ArH), 7.34 (s, 1H, imidazole-H), 7.31 (t, J=1.8 Hz, 1H, ArH), 7.25 (ABq, J=8.4 Hz, 2H, ArH), 6.82 (ABq, J=8.4 Hz, 2H, ArH), 3.82 ((ABq, J=13.4 Hz, 1H, ArCH₂), 3.24 (m, 5H), 3.00 (m, 4H), 1.97 (s, 3H, CH₃), ¹³C NMR (500 MHz, CDCl₃) δ 171.9, 147.2, 134.1, 133.5, 131.7, 130.1, 129.8, 128.9, 126.0, 121.1, 120.3, 119.2, 68.4, 64.1, 43.8, 40.9, 20.8. MS: m/z 600 (M⁺).

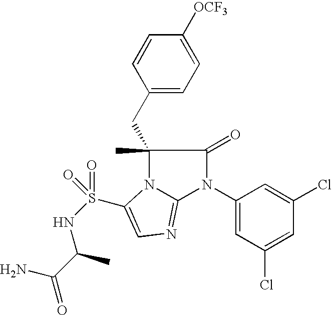

mp 96-99° C.; ¹H NMR (400 MHz, DMSO-d6) δ 8.40 (Bs, 1H, NH), 7.64 (s, 1H, ArH), 7.46 (s, 2H, ArH), 7.44 (bs, 2H, NH2), 7.16 (ABq, J=8. 0Hz, 2H, ArH), 7.00 (ABq, J=8.0 Hz, 2H, ArH), 3.75 (m, 1H, CHCONH₂), 3.77 (ABq, J=12.0 Hz, 1H, CH₂Ar), 3.29 (ABq, J=12.0 Hz, CH₂Ar), 1.97 (s, 3H, CH₃), 1.22 (d, J=8.0 Hz, 3H, CH₃). MS: m/z 605 (M⁺); Anal. calcd for C₂₃H₂₀Cl₂F₃N₅O₅S: C, 45.55; H, 3.32; Cl, 11.69; F, 9.40; S, 5.29; N, 11.55. Found: C, 45.56; H, 3.01; Cl, 11.54; F, 9.79; S, 5.29; N, 11.41.

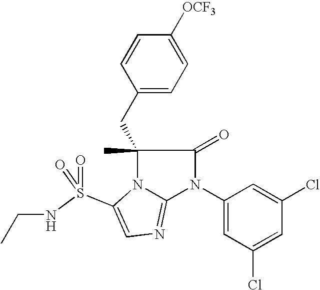

¹H NMR (400 MHz, CDCl₃) δ 7.39 (d, J=1.8 Hz, 2H, ArH), 7.36 (s, 1H, imidazole-H), 7.27 (t, J=1.8 Hz, 1H, ArH), 7.00 (ABq, J=8.4 Hz, 2H, ArH), 6.96 (Abq, J=8.4 Hz, 2H, ArH), 3.88 (ABq, J=13.4 Hz, 1H, ArCH₂), 3.26 (ABq, J=13.4 Hz, 1H, ArCH₂), 3.15 (m, 2H, NHCH₂), 1.99 (s, 3H, CH₃), 1.20 (t, J=6.8 Hz, 3H, CH₃). MS: m/z 558 (M⁺).

To summarise, the subject of the present invention is a process for to prepare a compound according to formula I as hereinbefore described including all preferred embodiments, said process comprising step c) of reacting a compound of formula XVII to a compound of formula XIX, including all improvements of step c) as hereinbefore described, step d) of reacting a compound of formula XIX to a compound of formula XX, including all improvements of step d) as hereinbefore described, and step e) of reacting a compound of formula XX to a compound of formula XXII, including all improvements of step e) as hereinbefore described and optionally step f) of reacting a compound of formula XXII to a compound of formula I, including all improvements of step f) as hereinbefore described.

Step c) with all the improvements as hereinbefore described and which concern said step is also a subject of the present invention, be it as part of the overall process for to prepare a compound of formula I as hereinbefore described, i.e. sequence of steps a) to f) or be it as a separate procedure for to prepare a compound of formula XIX from a compound of formula XIX. Details, relevant for this aspect of the invention (i.e. step c alone) are also outlined in the claims in context with the overall process for to prepare a compound of formula I.

Step e) with all the improvements as hereinbefore described and which concern said step is also a subject of the present invention, be it as part of the overall process for to prepare a compound of formula I as hereinbefore described, i.e. sequence of steps a) to f) or be it as a separate procedure for to prepare a compound of formula XXII from a compound of formula XX. Details, relevant for this aspect of the invention (i.e. step d alone) are also outlined in the claims in context with the overall process for to prepare a compound of formula I.

Step f) with all the improvements as hereinbefore described and which concern said step is also a subject of the present invention, be it as part of the overall process for to prepare a compound of formula I as hereinbefore described, i.e. sequence of steps a) to f) or be it as a separate procedure for to prepare a compound of formula I from a compound of formula XXII. Details, relevant for this aspect of the invention (step f alone) are also outlined in the claims in context with the overall process for to prepare a compound of formula I.

We claim:

1. A process for preparing a compound of formula XIX, said process comprising reacting the compound of formula XVII with a compound of formula XVIII and an organic base to form a compound of formula XIX: 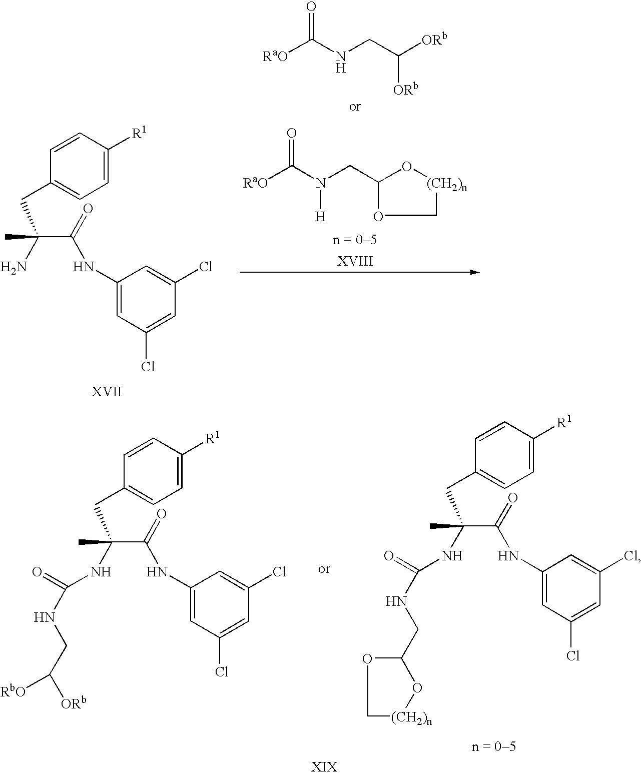
wherein there is no organic solvent present but said organic base serves as a solvent for said reaction;
wherein R¹ is selected from bromo, trifluoromethoxy, cyano and pyrimidin-5-yl optionally mono- or disubsituted by NH₂; and
wherein R^{a} is aryl and R^{b} is C₁₋₄ alkyl.

2. The process according to claim 1, wherein the base is selected from the group consisting of triethylamine, diisopropylethylamine, N-methylmorpholine, pyridine and trimethylamine.

3. The process according to claim 1, wherein the reaction further comprises crystallization of the obtained compound of formula XIX.

4. The process according to claim 3, wherein the crystallization is performed in a mixture of alcohol and water.

5. The process according to claim 4, wherein the alcohol is selected from the group consisting of methanol, ethanol, isopropanol, n-propanol, n-butanol and tert-butanol.

6. The process according to claim 4, wherein the mixture of alcohol and water is at a ratio of alcohol/water in the range from about 3:1 to about 1:3.

7. The process according to claim 3, wherein the crystallization is performed after previously adjusting the pH-value of the product solution with an acid to an acidic milieu.

8. The process according to claim 7, wherein the acid is selected from citric acid, tartaric acid, oxalic acid and succinic acid and the pH is adjusted to be in the range from about 4.5 to about 7.

9. A compound of the formula XIX: 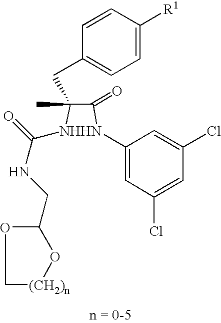
wherein R¹ is selected from bromo, trifluoromethoxy, cyano and pyrimidin-5-yl optionally mono- or di-subsituted by NH₂.

10. A process for preparing a compound of formula XXII, said process comprising reacting the compound of formula XX with a compound 1) or 2) of formula XXI, in an aprotic organic solvent to form a compound of formula XXII: 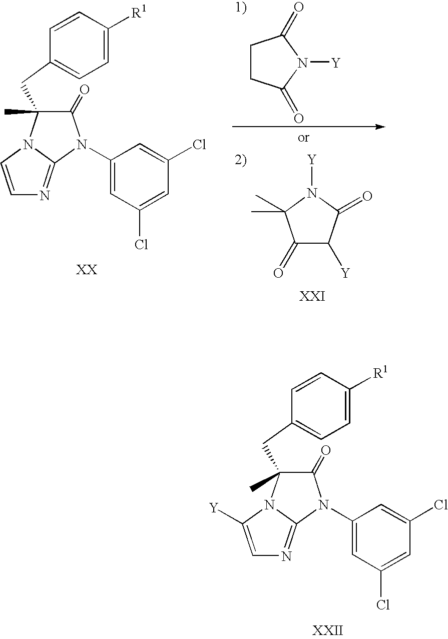
wherein the compound 1) or 2) of formula XXI is slightly soluble or not soluble in the aprotic organic solvent; and
wherein R¹ is selected from bromo, trifluoromethoxy, cyano and pyrimidin-5-yl optionally mono- or di-subsituted by NH₂ and Y is a halogen.

11. The process according to claim 10, wherein the compound 1) or 2) of formula XXI is added in portions to the aprotic organic solvent containing the compound of formula XX to obtain the compound of formula XXII.

12. The process according to claim 10, wherein the aprotic organic solvent is selected from the group consisting of isopropyl acetate, ethyl acetate, n-propyl acetate and n-butyl acetate.

13. The process according to claim 10, wherein the compound 1) or 2) of formula XXI is added in portions to a solution containing the compound of formula XX and isopropyl acetate to obtain the compound of formula XXII.

14. The process according to claim 10, wherein the amount of compounds having nucleophilic groups is reduced to be equal or less than about 3000 ppm.

15. A process for preparing a compound of formula I: 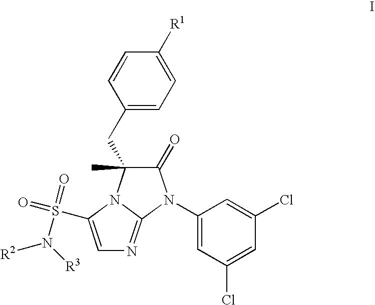
wherein
R¹ is selected from bromo, trifluoromethoxy, cyano and pyrimidin-5-yl optionally mono- or di-subsituted by NH₂; and
R² and R³ are each independently selected from the group consisting of
a) hydrogen; and
b) a C₁₋₄ straight or branched alkyl group, optionally mono- or disubstituted with moieties independently selected from oxo, —OH, NH₂ and —C(O)NR⁴R⁵, wherein R⁴ and R⁵ are independently selected from:
(1) hydrogen, and
(2) a C₁₋₄ straight or branched alkyl group which alkyl group is mono- or disubstituted with moieties independently selected from CONH₂ and OH;
or
R² and R³, combined with the nitrogen they are bonded to, form:
(1) a pyrrolidine or piperidine ring, each optionally substituted with the group —C(O)NR⁶R⁷, wherein R⁶ and R⁷ are independently selected from
a) hydrogen; and
b) a C₁₋₄ straight or branched alkyl group, optionally mono- or disubstituted with moieties independently selected from oxo, —OH and NH₂;
(2) a morpholine ring; or
(3) a piperazine ring;
or a pharmaceutically acceptable salt thereof,
said process comprising reacting the compound of formula XXII, wherein Y is halogen and R¹ selected from bromo, trifluoromethoxy, cyano and pyrimidin-5-yl optionally mono- or di-subsituted by NH₂, with a compound of formula R^{d}MgY, wherein R^{d} is C₁₋₆ alkyl or C₃₋₆ cycloalkyl and Y is halogen, sulfur dioxide and N-chlorosuccinimide, followed by a base and a compound of formula XXIII, wherein R² and R³ are as defined above for formula I, in an aprotic organic solvent to form a compound of formula I, without isolation of intermediates formed during this step: 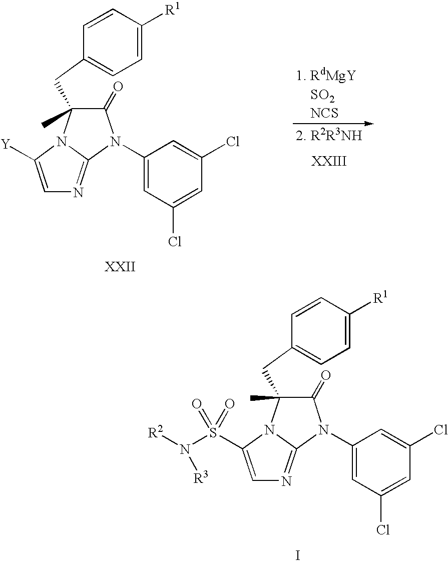
wherein the reaction sequence comprises sub-step 1 (N-chlorosuccinimide oxidation), sub-step 2 (sulfamidation) and optionally sub-step 3 (crystallization),
wherein the N-chlorosuccinimide used in sub-step 1 is dissolved in a solvent that does not interact with the N-chlorosuccinimide.

16. The process according to claim 15, wherein the solvent used in sub-step 1 and/or sub-step 2 is selected from acetonitrile, propionitrile and benzonitrile.

17. The process according to claim 15, wherein the base used in sub-step 2 is selected from alkali or earth alkali hydroxide, or an aqueous solution thereof.

18. The process according to claim 15, wherein the compound of formula XXIII used in sub-step 2 is in an aqueous solution.

19. The process according to claim 15, wherein the crystallization of sub-step 3 is performed in a solvent mixture of ethylacetate and methylcyclohexane.
